SEQUENCE LISTING (9 sequences)

SEQ ID NO: 1 - DNA, 26930 bp, Homo sapiens
gggctcttgatgcaatctatatgccaatctacttcattagttcttcttttatttacagtttggtaaagaatatgggtggagctgttctgggctcacttgcacacatgtccaaactgctttgaaaaaggaagggcaagaaagagtggtatccaagttggaatcaggcaggcatttcagatcaagagacgaactggaaagggaacatctgttagataccctgggtttgaaggcagtctgtgtaagttttcatatctctgagtgtgtgcacacagtggagagggtggagcctgccatcctcaaatctgaaaagattgagagatttcagagggcccagatgtgccaaaggtcagagggatcaatatacaggccctaccacggaaaggcggggaaaaggttcgaatagaaaactgctgcagaagggaagccactgagaggtaagggagtttctgaataattaaaaagttaagaataagcaaaaggaaggaggtcgggtgggggataaaaaaaagcagttgatgtggtaattaagaatttggtgggagcctgggcaggtcacctcctttctcagatcagagccccatcagaaattctttcaagtgtccttctgcgtcgccaaagatgacaacagcaaatcaataagtgcttgaaatgaaaggggatgttgactagcccccaggctacagatttcccgccgccagccttttctgaactcctatagcgtgcctttgcaccgcctctcttaagaagagctaccttttattcctattctcaggacgaaggtaagtgctcagttagcatatctattaaatgtcagctttggttccagctctccgtttgcgcggaaagctcactgccatagcgcgcccagcctgccggaggggcagacagaaaaagcaagcttggcctggcgacttgcggggccacgcacctccagggctggcccggagtcttccagagtttaacgctcctgggttagaactgtaaggtcccggtccgagcaaagggcctgagccaccgtagccgtgggagcgttccttccacttgaatgcactcactcacaaacaagcacaaaatctttttaacagcagaggagaaagacccctgctccaaaattaaagctgggaatcaccggaaactccgttttgagtgcgagatattggtctgttacctttctaatcttcatatccctcctgtaatatgtcttgatttaacaatctttccagcgcccagcattgcccggacgttttaattgggtaattcattagtgagtcaatacaggcatttatatattcttttagctcaagtggttaagtactaatttcgaaatgattataaaacaccggaatcggcaacgcatagtaattttaatttatatgcaaatcaattggttcatcttaaatgccttttttaaaaaaacaattattgtattgtagcatcggaggcatggatcaaacctctagaatagacaattcggaaacaagacctggactaggaagacaatttagaacagccaacaaatcaataatgtttgaggcagttaaacatcgctagccattggtatttacatactcgcttgttgtcagataaggagctggggaaattgcttgccagggttgagatcataatccagagtgaagaaagtaaatggtagcacacagcccgtcacagcgggctctgaaataatactgtacctttcccaaatcctgactcttgggtgacagggagttggcggaggtcaccccacatttgtccacggttttgccccaatttgatcacgaaattgttgttctccctgagctttccaatttgattaccattctaacggttctgtcacttgtctcaacatattggggggaggagtgtaattgagattctcattaaaaattatctgaacccacttagccagcactgtttcatctaagcttagttttatgggctgtatttaattccctgtgccccccacacattaaaatcagatcatcaaaatgtcggtaggaaagggtgaaggaaatggtccaatgctccagtttactggaagactattatctttagacatagttcaaaattttgaggaaataaaaaggatatacgctttggggggaaaatgttttaatattctagaatgggggtattactccccccattccagagaatccgcactggagttgtttatgtaaaaatgtaacatccttgaaattcacagatacgtaaggttagtgtctccccttccccaggctcccagtccaggcgatctagccctaaaggagctagtacctttgatgctacaatcttgtttacatctgcagggcagagaattgcttgctttgcttggacgctccctccacccccttctaatttgaagtaatcggaatctaaatacagtcgccaaggcccgctcttcctttactgctttgacaagggaaaaacctgaaatccacgtcttaaatcagctcggtggtttgtagccccccagcaccctgcttctacgattgcatgcctaatgtattccctggtgattctgggcattaattagttgtttaataggagtatgactaaaaatgtaaaagaaggattaggagcgtgaaacgtatgtccagctccttccacacactcgaggagggaatgagaatcattctgtatcttctatttctccaggagccatttgcatttcccaccagctgctcacttcagctgcactggcgctgggcaaggcgaggacccaaaagctcagcgcagtgtctgcggcggccgggactggggttaaccagcctctggcgggcgagactccagacagaaggggggcgagaggaacgtgagcttcccgagccccttcctctcagccctggtttgcaaacctctgaaacctgaaaggggagggagttgcacgcgcgtatctttgcgtcttttcagcgcaactcccttccctctccctgtgtctctccgcggatctctgaatctttctgtctttggttttctcattctcttccaacttttccatgagattgcctatcctcgccaccagctgaaggcaaggccgttctgctacgagcgcctcttaatctctacaaaatgaaaagaaaaaaagggaggattattagcccattactcagaggaatggggaggctgcaaaaatcgtcgatgggcagaggtgaagatgtctttctcggactgcactttccggtgtcctgtaactagagttcagttgtgggacttgttgaagaaatttgattttcttgcctcggcgagatttcaaaaaccagaaatagaaattctcagagtcagagaggaaatacaattaaacagcacgtgggcattttccccctcatttctctccccttaaataacactgctttgagtttccactgggtaaagagagaaagtttgagttttcacggatgttacgtggaggttagaaatggcttaaaatgtagatctctaatcagttttcttcgtggctgaagaggctaaccctttccataaaatgagtccatctgtcgactgttagctatttcaaagtgaagggatttagcactcaaaacaaattgagcaagtttgtttgcctgtttttactgctaactcaaatgaattcaaaacacggagtaattcaagaaaacacataacatgttccagacagcccccaaaagtagggaaagcccagcacctatatagtgactagggttagttttaagcgccaagcttttttaaacgtatctattttatgcacattctcccgagtcactatatatttctaaaattgcgagtattggtatattgatttaggaagagcaatacaacttttagagggaactttattctcaattagggaccaaagagatgtctttttaatagcgggcctgagttttgctctcaagcaggaattaatattggtgggaaaatccgaatccaggagcaatggctgtgttccggcactttccaaaaacatacattaacaggatgcccttgagattgaaaaaacattgtcccatatgcctggcagaagccttcacacctggtcctccaggcgaattatatttatagtccttccactcagaggcaggacagagccaaaatattctgctcactaccaaaatacacatctttgctcaagtcaagaaatcagaaaatcagggttcagaagtaaggcacacttttcgagtgagaatatgccctgtaatttcacatactctttgctttgcaggagcaaatgtggacttgagggaaactctctcccccacccccacttctatcccgtgcaatttaataccatcctcgccaggaaccttaacctcgtcattttaaaaaatgagatatccgtgacccagggtgaacttgttgaatgtaggtacagcagaggaaattctagactctatgagcgtctgagccttgtccagtgcaaacccttcgtgaacactgggtcagtgcgtggccgtgcccacctgtgcgccgacactctcagcatgcctggtccacccgccttgacctcgggcgcggtgtcccagctaagctgggcccagcgtcccggccttccccagctgacaagcctagctcgttcgctcccggctgtggccctcccaccctctcccactagctcactccattcttctagatttctcttcactcatcctctcccatccccaccgcgcccacctccactcccgccctctaccggtctctcactttcctccctccgcagtccctctttgctgtgacctctttcctcaactctgcaggcctgaaagaaggtcacacacgcacgctcacacccacactccacacgcctcgtcccaaacaaccccatgaacattgtcctttgttccgtctcttgggccactttccctgtcgcttcctcccagcccgtcctgatttgctccccaaaagtacgtttctgtctccccgctgccctggcgctccccctttgatttattagggctgccgggttggcgcagattgctttttcttctcttccatcccatcctcccttctggtcctcctttccacagtgggagtccgtgctcctgctcctcggttggctcctaagtgccccgccaggtcccctctcctttcgctctcccggctccggctcccgactcttcggcccgctggcatctgcttccctcccctgcctcgtttctcgtcgcccctgctcgctccccccggcgctcgcccgggcgctgtgctcgctcctggatcgccagccgcgcagccgggctcggccggccgcccgcgcgccactgtgcagtggagtttggtggaatctctgctgacgtcacgtcactccccacacggagtaggagcagagggaagagagagggatgagagggagggagaggagagagagtgcgagaccgagcgagaaagctggagaggagcagaaagaaactgccagtggcggctagatttcggaggccccagtgcacccgtggactccttcggaacttggcaccctcaggagccctgcagtcctctcaggcccggctttcgggcgcttgccgtgcagccggaggctcggctcgctggaaatcgccccgggaagcagtgggacgcggagacagcagctctctcccggtagccggtaagtggaggccatctatcccgcagggatgtgagataatgcgagtctggaaatttgttccacttcggagaatcttcaccgtaggtgatttgtggcttttggggctaagtttcgcccaaggtaacgcagtcggcaaacagaccttgcaaagccctgttcctttcgtcccccgccacagacactaacaatctacagggtgctgaagtcgagagggaagccagaccgtggctggcatttaaaacgaggtatcttcccttaaatctcggtgccaacactgcaggaacaaatcctcgggccaaggattagcattctcaagataaagggctgggtacaaagtttcagctactggaagattagcccccttcccattgttatccattgggaaaaaaaagaaaagaaaaagattccatcttaactggcagttagtgacctctcaggcccaagcgaattacctgggagccaggcctggatgccaagctctcaccatttctttggattgtaactcctttaaattgatcaccagtcaactccaatctggcacttcaggagatacactttaaatggatgcagagaattattttccagctggagattaagaaaaaaattttcgattctaaacctccgaaatatgttcctcttttccagtttaaccactttactttcttaagcaatttagaaatcaaactatcataaggtggtgtgatttttttttactcttttgtgtgagtattgtcttactaaactaaacggaaaaaacttttaccattataaatgtaaatatcagaattcatacattctaaaatatttttatgaaaaattaatctgatttaaagaaatttccttgcatttgttttagtctatcaatcaaaactaaagatgcttttatcacacaaaatatcattttggcagaaatccatctaaaattcaaataccaataatatcaagaaaacaaagcacataagcaaaataaattgaagatttttgttgatgtaacatgagcatacaacatttcaataaccaaactttccctaaaaaattaaatagccacttcatttgtggaatgttttactttaactcagcaaaattacacttaaattatttaggtgctttgttccttaagttaagcgtgtttgtcttcaaatgttcctaaagcacttatattaattggttgtaaagaacgcatacacatggtaaaatacagaactgaactgagcagtattttaatttccttaaataattacttactacaaattaatttactggctaatttcacaatttagttcatttaaaacacatgttcctgtgctgtttatttttaaactttccattaaagattttgttatggggtaacaaagtgtatgaaaaggggggaaatgtgaaaggatctgggattattcgaactgtatttttcctgcactttcagtcttgcggtagtcatcagaaattattttttagcaaattgttttatttcttagggcttgcctgcctgctttgccatggttcctcgtcctccgttagccgtgtagtgctttttgtgtgctcacaatataaaacccaagttggccaaaacaagagtccttggcatatacattccaactagaacatgaactttgggggtgagaactacctcccatcaggaaaagtctcccatctcaatttgtgagattagccattgaagccagttccgaagtctggcagccaaatttctcacagaagacttgtcttgatagggcaagtttaaggatcagcaggcgggaattggaggtctctttttaaaaaattatcttccccagttatttagactcagttcttctagtaggcctggtcattaaatgaagcataaaaatgcaagtctcaaggctcattttgactgcaaaataaatctccaagtcacaaggacatgtaggagtgagctaaggaacacgccttgaccttcttttcagtccttagagtggagctctatgagttcttgaagatttgttttgtattgctttgtttggtcttcagcactgaagcacggggaagtggggggaagaatgtgtaataattgactgactttacaccaagcaacgcaatctttttcttttgtatatttcattctttaaaaaaaataaataaataaaaactatttgcagttaccatctgcagtgctccggctaccagctaataatgcagccagttcagacatataaaaaaaaaagattatcgaaatgatgatgacatgcaaatttcctccgaaattatcataagtaaacatttgaagtctggactaataaaatcccatctgtgttacttcatatcgagttagtagaaagctgtgataatgaattttgtaatatctcacgaacagacatctcaatcagggactaatcctgtgattttactgcagaatcactaaatctggagccgccaaactgctacttctgggcccacgggcccacaaggatcgaatcggcagagtccccgcccgcgttctcgctagcgggtgggggaaccgcctggccgtccccaccctggatccccacgccacagcgccgggcagcccctcctgtaggcagcgaccttggccagaggctccccagggcccagctcccttcaggagaggccgagacgcagggaaacggtactcaggccagaggcaggcccgcagctccctgccccgcctctgtgcctccgccaacccgacaacgcttgctcccaccccgatccccgcacccgcgcgaagtgggccctccggtcgtcggcgtaccctggttagcgtggagagaggcaggcgctgagatcgaaggggcctagggagccctggaccttcttttcttgtctttaaagcaaccgcggctcttctacccacccggtggagcccctcgagacccacctctcccggcttgcctgtggcagagaagggggagcgcgttaaatgcttggctcgctgcgttgtggttgaaaacgtgaaaaagatttggctcgcccgggagagaaagggggagaactgggtagcagttataccagagctatttctccgtccttggcgggcagtaaaccctccaagaacgtttgccctgctctcctcagtctcgctcagtccacccagtgtctctcctctgcgatcttaaatcatactttagggtaattatttgtagtaagtaaataaatggccgggttagtatctctaggagaaagtgtggctaaatatggaaaagtggctcctgatggatgagaggcccgaactcagctcgctcctgaaacaccctaggccaagagcccgttcgtttcagaattacagaaaaccgagggaaactgctgtctaggacaggggcacgttggcgctgatgttctacaaatgtttaccgagctccaactaatggacaagcactgaagggtggtctttgcatacagcttcccaaagagaaaagtcctctccacccacccactcccgctgccattgcgttcagatgagttcttaaccccggcaccgagattcttgaaagtaggtccacagcctccccagcacactgtggctttatagtcctctaacctctgggcacttttgcggcaactctggagggagatcccctcttgataaataaatgtcctgggcccgaggctaggctggagatgctgctgcacagccagaggctgtcaggtcggaaaaacacgcctgaagcctagcacacagtaggcgcctaacagctagtgtaacgtagtctcatctgagccctgctcactcgacggccgccgcttcttacagccttccttctcttctgttttgcagataacggggaatggagaccaactgccgcaaactggtgtcggcgtgtgtgcaattaggtaagaacccccttctcctgcccgggtcatcggacgggaggccgcgccacgtgagggcggcaagagggcactggccctgcggcgaggccccagcgaggggcgcttccccgaggggccagcctgggcaggaaggaaatcagaaccaaatcgccagtggcctctccctgtggcggggcggtggactaggaagcagcggcgcgctgtgtaccgaagccccccagcctactcctcccggctggaaccgccggcaaccggggaggcgcagaaagagtacgccatcctgccccgggttgccagagggtccgggggacggggatgccgtcagctctttcctctaactgggtctttgctctttgtccctctttctcctccggcctgcctcgcccctccccctcctcccgtccccggctcctttcggccgcggtccgggacgcctctctccgcacgtggggcgggcgcgcgcgtggcccaggcgtgcagccggcggccgttgaatgtctcttctccaaagactccgaaatcaaaaaggtcgagttcacggactctcctgagagccgaaaagaggcagccagcagcaagttcttcccgcggcagcatcctggcgccaatggtaaggccgggagggaagcgcaggccgcgcgctgggcacccgcctccgggactctgggcctcgggcgaagcgcaagaaggcgaggccgccagacctgacgcgcctgctgcttgaacttagacactccgcctctgggtgggacgggaagcagccgtcccagggacgttaattcctttccccaaattatactgcactcctgagacccaacacctctctctccctctcgctcgctccctgtggtctgatcccctgcgcacgctccagcaaacctcggcgtctaggctggcgtggaaaagtggtccaacagcgacctctgtcgctcgttatatcccgctgcgcgcagcaccaccagggcccactcagtcactcaggctctcagccacgccccacccagacttgtgggtgcgcggttcatcgcgggaggcaagcaagggaaacttgagttggcgaaggtttgctttggctggttgggggaggggcggggggccgacaacatccctgaagagctggagggcagccactgtgcttagcagcccagggtagaatggaggtttgcccctgtcgacgcgaacctgcctgaagtactggttgccaggccttgggttttggcgatgtcgttgcttgattggctggtttcactctggaggaatcgagggacattgccagaggaggtctacaggcttatgtaaaaagttaaaaagtctccaacctacgccacaggcctttcctgaattgaaacttgttctatggggcggaggggggggtgtaagggatggaggagggaagatgcctttcttttcaaatacatggaaaaaaacccctcaaatttactgttcctctattttcctggctccgtagtaaataagtgcctagccttaggaggctattgacctttgataatgtgagcagataaagcccccccccccccacagccctcggcccctaaccccctctttccggattaaagtgtaagaatacaaatgtaatatgggatggaggggggcgatttgggaccccggtcaaaaaaacaaaccgtattactaagaagaaaacaaaggtcttgcattggagtttccccgtgaatctgagagaaaatgatcatttgttgaaatgaagcgtctaaagcgatccagtgcttcacgcccggacactgcactacaccgccagtcgccctgctgggccagttaaacgctcacttgtccgggattaaccccatggggttaaatgggggcaatgcagagataacgtcgcgtgatttctgtcatttagattgtgttaaattcttcttctgtttgataatcggtagtaaaaataaattattagaccgtagtatgtttgggatatggttaaaaatcaagagcagcgatgacttctggggagaatgctttgcgcgggctcagccctggctccggccagactagaggagctccccaatctcgcttcgcgcggggcgggctcgcagtcgccaagccgaggctgacatttcttattgtgctgggagccagagagacgcaaaatgtctccttcccccagtccctaccccaggtttcctagacatggggaatgcattctgaggacaggtggagaagcctacggtaggatggggtcctcgtaggtgagcaggaaacggctaagagcagaggagttctgcttgcgccagtcacaagccgcgcaggcgcccctggtcgttcgcttttgataactagcacataaagaactagaaataatgaatgattgctttcttaaccattactttcaggcccgcattgttttagtgcacgtgaaaggctcttcccctacactcaatatgtctttttctactttttgaccgaaaagaaaaattgctgcctaaacacgtttaatgccattaattaagaaaaggcatgtaatgggaagaaatgctgaaaatcttgatttaattggcttttaaggaactagtagacgacaaaaaaaaatcacacgagtgggcaaagctatagcactgctgaaggatagagcacctatccttccctgattttaagttaacttatggaatatccaaagtcctggccacagcctgcttgtaaaacaaaaggatttatttcttgtgtttttttaaagtttttctttgcttttaaagagaaaaaaagttcacaatgacatatgacttcttcaaaaggccgtgatagtctattacgctaccctctccgcctctgcccccaacgccgcccaaaaataccatgtcctcgttaaagactaaacgttctgtataggcagagtccacctctaagcagtccaggctctgccttccttccctagtgagtcccattctccttggtatccattgggcggatgcccagcctggatagaaccccgaaacgggggtagcacgagagcgactggagacccctaaaagccagaggtttgagagagggtggacgcagctagcagaagatggtgtagaagccagctgagaacgaccccctagagcaaagagacctttctttggcttttcttgctttgggggtcctgaaaggaactcataaaatggtcttcaccctcaggaggaggacggactgaccctcctctgtcactggcttaaaaagtttcagggcggcggctctgggttccgtgctgaaatcggactgcactgcagcccctttggacctgacgcctggctttgcgtcccgacaaggggcgggtacttcccccggcctcccccaggaacgcattaattgttaaatagctttggcccagtggatgggctgaaagtgttcgacccaagtcgctggtgtgcacagacttcctccctctgggaggtgggctccatggcccgttgtggcacccccagccgcgacacacaccttcacacgcggcagcagctcggtcccaaccttcttcgaaggacctgggttaaccctggcggccttggcggccgcagacccctctccgccgccccgcccccgcgcctctcattcaatcagcgaatgtttgcggagcatatactacgtggactcctaatgtaccccctgaaagcaaataatacagttttcctcgccgtcatgaagggattttaatcctaacatggacaccagcgagatcagcccagaccgtctctagcaaaacgcaaaatggtggtgcgtggggtggtgaccaaggtcctgagccttgtcagaaagaaggggatgtgtagagaaaggtggagaactctagctgtggctagcgcggaagggacaggtgcttgccgaagggggcatgaggcttgaggaaaaagcaacgaaataggggtaaggagagttttccattttctttcctccgcccgacctccgccatcccattctcccctcccctcccccaccccccgcgccaatcaaatctgcagctcacttgaaaggtgccccgccgcgttgtggtttttcacccccaggggaaattgtaccagttgtccgaaagtagtcagtccctgcggatccctgcccgcaaaagtggtcttcacaggtcgcgttcctcgctgctgactcggtacacaaagtttcttaaggctggttcggctgttattcctcaccgcccgctgctaatatatgcagcagttgttagagcggctcgggggaaaaggaaatgtataacgaaagcttattcgtgagcaggaatatactaatggaacaatctgatgtcttcttaatcctatgtaaaaagctttgtcgtcttcctaatattgactgaatgggtaattaatggctcttcatctaggcgaataccctgtaatccaagataggcaaaagacaacaagcccaaggtagaagacaaaaggcccaactgcagcggcgtttgcccgcctcctaccccccagggtctctgactaggaaagttttctctcagaggagaaaaaggcaggagtgggagaatatatacctatcatttcggggctagacttcaccgcagcacctgaccacccagctcagttttctgttactctgtctttccacctccagtccttctccctgcatttttttttcttcttaactcctctaggatgacctcctaccaccaccgccattatggtcctaataacctttccccctaaaccccacatctttcacttcagcaaccaatgaggctgttttctgatccaggaggagattcttttcttttagaacccaatgcgtagagtctttgagaactaaagtagttggtaggggaggaagaaattaatagaaagggagagagcatacagaattgtgtgtgtatgttaaagagcgaccaggaatgacagagtcaacttctttgtgaggatctgacgggaagagtgtccaagactctaccagcatgtttttaacaggctgacattttaatttaaacctttagaagtaatatattacttgggttactacaatgaggtgggttcctttttttttgtcagctgacagcttttaaaatattatttcgctagggaaataaaagcttcatctcagattataggtgggtatttttggatttaggtgatttatggtcaccatgacaactaatgctgaatgttagctaccagcatgtctgggagagagaaaacagaaagaagggagagcaaaagaaatagaaaagggagatggacataagctggagagggaagaaaagagaaaaagaggaagacagatgagtgtttaatccaactgttgtttaaaaaagtggcgggggggtgggacttcatttagccccttgccacttccttctttcctgacctggatatctatgctcaatttcatattccacccttccttccccttctccaaacacatgtgctataccattctccttattttacttagttcggcaagtagttgctttctggagactcagtgacacctaggaaaactgtggcagtaacatgcaaatgtgaggaagcattaacagcatgttcgttgagtgattttagcaaatgccccctcctctaatctctttctcttctcttccccaggccactgtgaggtggtaactcccactgccatctgaacactgttcccccaggtagttaccccaaatctcaaatggttgagcagctagagctgtgggttggaaaaatgggtaccatttgcagggacccagagagggtggctgttgctcaatatatctacagacttccaattcagaaaataatcttccccttttgacaagccagagctttttaaattccatttaggaaatggggaaaaggacagctacagtgaagcttttaatttctgggttatttggttccatagctatgaggggtggtgggtagtggactgttttcagcttggtttgtatgcagagaaaagccagatattggagggggtggggcacttctcgggcaggatgcaaggtctccatctgacctctgcgccttaccaggagctcacacactcacgcccatcacgtggctccaagctgagtccaggcgggcctcgtccttgagctagcttgggcagggcaggactcccacccgtctaaggcctaacagctcagggagatgtctaattaagccatcttctgggtgaactctgaagacagactctttccaaaagtcagagatcacttggttgagtcccaggccagattgatacggagagtttggcggcacagcccaaccgctcactgccatggccagagggactttgcacaactaacactgaagagtgtgaaattaaataagactttaagactggtaatcggtggcaaataccagcacaaaacacggctgaccccatgtcacacatttccctcctctagggtctttctttgaaagaataagcaagaaaccccaatcgagacaacccctgatgtctcccagactcaaaacctcacgccggcactgggctttccttctttgccccacgtgagccatgcagcacctctagtttccctaccaggatcccaccaatgttctccgtattggaaatttctgtgttagaggctgaactcatagtaatttctaaaaccaattaagaagaacttagccagaggtcacagtaatgctggaatcacaaaatgcataagatttattttcttctggcccctttctcatccatgctgcctatgcctgtgcacccacaagtcttatgtacattaaatctccaaaatcaaccaccaccatgccacagagttttcactggacagtgtttcttagttcccaccacatacctccttttccccatgcagacttatcatgttggtgtcctgccacacagggggcctgagaagaatgtcacccaatcgccgctgctgtgagtgtgtaaagtgattaggagattaggagaatgttgaaactcctgctggaaaaatgcaaagaaaatcctcactttgagtcagttgtttacagagccagtgtgtgtgtgcgtgtgtgtgtctgtaatataaaatggatgtgaatatatatatacaaatagatatggttttgcttttactttaatctgaattatctagataattgtctttattcaccacttgacttcaatgggtccacacaaattaggacaccttatctttttaggcatctaggctgctgctgatccccagtgcttccaacatctcgcacacgttggcactatgaggagcagtcacgtgcccttgggtttttcaaccactttggaggctgattgaggtccttcatacatgtatatttgtcgtgatgaaagttccatcggtagagtggagccaccagagcttttatcaaaattctgtgggtttatgagagatgggtttagaaatctatatggctctgtggggcttctcggctttctaaaataaggcattaacacccaagcttccaaaaatatttgcagctctggggtttgaatcttgaaaaacaaggagtgaggggctgtgtatactaactacagtggagatttttttcattctttaatgtgatggagtcccttcatgaaatgaagctttaaggggcatggtattgtggggaccacagctattctgaggtttaaaagaagaaactggaatatgattagtaaacacattcagcagaaaagagctggattcttattgacttagttataggtcatcggctggcagtgcaatgggaggaaatatttactttacacatatactttatgatcttgggggaattagaggaaattcaataagaaaacggctagaaacatttaaaacccttatttaaaagacttaagcaaattagagtcttatcagattaaaaaccactacaaatgtaagagcattgtcttcagtgaaacgctgtggggtctgagaaggagattctccgccaaatctccgggataaaatgcgtcatttaagcaccagataatgagcagaatgtaaattaatttaaccttctttaccaacaggctgctagtgtaatgtgtataatttagtgataagattgcaggacctaatatagctggatgtatgagcctcagctaatgcagacctgtcacatgaggatgtgttttactctgagcaggtgtctgtatgtgtggaatggggtaaagtggaataaaaggttaaaagcagaaatgctgatttaaagcttactatgaagaaattcctcccttgcagctaaattattcttaaagtgggatgatactggtgaagaaagactgaaaaacaattctcatgtgcgtctttggactgcaagtttaaaatggggaggagttgcagatagggtttgggggtggtcagggcaaaggagagacacataagttgcaaatatatttgtagtctgcttcatccactttgttccacatcgaataagtttcccaatcttgtgaacaaggacaaggagggagtgttttaaagatacttcatgctggcattgcaaatcattgactgtaatgtcaaacaaatacacattcagagatgataacactaactccatagtaaaacaatcgcccatgcagaaacccagaggagattagtttgtcctctccagctgacctatgctgggggacaaaaggactttcaaaaattattttgaatatgtttggatttctttctttaatttctttggaaattaaatttgcttggaaacagtgctataaagagttgatgtctccaaaggtgattttttttgttttatataaataaggttttgcttttgctagttgagcgcagttctaggcttttcgcccttagctcacacacaccccttctgcctgcttggactttaatggctcaagacagccttgagctcactgggaaaagaaaatgactgttaaaaattatccttgaaattggttatttggcaacattcttaattgtatggaaattcattaaggcatatttcatatataattagctcaaggttgttgattctacaggctttatggatttaaatctgattgataataaagtaaacaagagagtcgaatttaaagcgtggctctctcgggttaggacgagcttaatacagtgtacaaggaatttgaaagatctaggatatgtgtcttaatcaacgttaagtagaatggataagctttcagcattttgaaaacgctgggttagggtttctcttctattgtgtgttttctgtctggggactaataagcatcacagagaacgtgatctgaggcgactttttattcttgtataaatccagagtgaaccaccaaacagttgttcgtttaaagtcaaggtaattttcttttgacgggtccatttgcttctcgatttctaatttattagcctgccttttcagggctctgtcttctttgcaattaaagcttcttcagattagcgcagcattcacttgacaggctgtttggaaaatttaagatcggagaggtgatttgttgctgtttttcaaattttctagttttaagtaacgtgtctcctttttatatggggtgggggattggaaatggatgtagtgagacacaaagagtgggtgtcttgttgatccttgtacctttctcttcttgaccattccactctcttctcccaagccttcgactcctagcctcatctcttcacctttgggttcgtactaaaagccggatcgccttgggctgggcaggagctgaattcccgggagcttgcctgtgtagacccagtgcgcacggcgaggcagtagcccggccccgcactgctgataggtgcaggcaggacagtccctccaccgcggctcggggcgtcctgattggtgcggagccacgtcagtcgcacccggagaagggtctgggaggaggcggaggcggagagggctggggagggccgcggcggagtgacgtctcggcaccaggaagcccgcctctggttttaagatgttaggccaacagggaagcgcggagccgcagatctggtccgtcgctcgcctgggtgcctggagctgagctgcggcaaggcccggctcctgttcgaccgcccgaggggtgtgcgtgtgcgcgttgcggagggtgcgctcagagggccgcgtcgtggctgcagcggctgctgccgccgcaggggatctaatatcacctacctgtccctgtcactcttgacacttctctgtcagggctgccgcgtggggggggggcgggcagagcgcggtcggcgttagctttccttattggaggggttcttgggggagggagggagagaagaagggggtctttgcccactcttgtttcgctttggagcttggaagcctgctccctaaagacgctctgagtggtgcccttctgcccacatcccatgtcttcgtttgcccgctgactttccgtctccggactttttcgcttgagccttccggaggagacgggggcagcttggcttgagaactcggcgggggttgcgtcccctggctctccccgcagcggggaaactccgcgcctagagcgcgacccggagcgggcagcggcggctacgggggctcggcggggcagtagccaaggactagtagagcgtcgcgctccctcgtccatgaactgcatgaaaggcccgcttcacttggagcaccgagcagcggggaccaagctgtcggccgtctcctcatcttcctgtcaccatccccagccgttagccatggcttcggttctggctcccggtcagccccggtcgctggactcctccaagcacaggctggaggtgcacaccatctccgacacctccagcccggaggccgcaggtaaggcgccgcgccgccctgcagacattcccgctcagctgctctgcgccacccgctccctctcgccccaaggaagtcagcccctccggggggaggcgtggtgggagtggtcgttcgcctggctccccgcagaacttccgggagccggaattttgactaccccgcatccctttagttctccctcgaccggcccggctcctggggcgctaagggcgcgagcaattctgccgccctctctattcgtaccctggcctcccttctgtttcctgggtcacaaaaatcccagcatcttgattcgaggaccttcagaggccgccgacctctgtccctgttttcctctcggctttcagctcccgaggagctccactcgttaggaaattgcctgaaaccactcagaaatgcccttcgcgaagaggcattttttttttttttttgggaaagggccggcgaacttcggtgcccaaccgaatccccacatcttttcctagccttcccaaaccgcatggaaatctgagctttctgcgagggggaggggggtctgtaaaccacgcgcgtgtgcgcgtcccaggagatttggtgtgtctgcgcagaggtgtataaatatacttgaaagcacaggctataaaagtgaatgtgccgctgcagtgagataaacatgtaaataaaacgtgcggcgctgggggaggggaggaaatggggcgcggacacccacacttgcgcctgcacaccccacaggcgcagcgctcctcgcggcccggagccgccgcgcgcaccctcctccggcgccaggcagcccagctcttccacggcttctgccgccggtccagttggcgtccgcgttgcaggtgggcatgctgacgggaaagtgtgtgtgtttcgttttcagagaaagataaaagccagcaggggaagaatgaggacgtgggcgccgaggacccgtctaagaagaagcggcaaaggcggcagcggactcactttaccagccagcagctccaggagctggaggccactttccagaggaaccgctacccggacatgtccacacgcgaagaaatcgctgtgtggaccaaccttacggaagcccgagtccgggtaggagccagcacggagtctgggagggatggggggaggatgttgtggaggtacaggccaagtagaccaggagagaatgtggaaggcagcgccgcctgggagggcgccggtggggcgcagctttgcaaaggcagaaggcctcgcggcggcctggttgcgagattacagttccctctccgaggccgacaggactgccgccctggctcaggctcccagagcggcaccggctcactgccccgccatcccgcgatctcacgagctgggctgcatgggcaatcccctgcacaggacattgtgttcctggcttgcagttgccagagcagagctaataaaatccctaccaggccaagagccgcgaacaggctccaacctgtgagcctttaacaaggaaaacccgccagagacacggaagagttggccctccctgggaaacctttgtcccggccctggcccagctttttccctcctgggctcgcgcttcttacaccttctttacggttgtttcggccattcaggtctctcccacacaccctatttcctagttttgtgatctccgggagcaaagttttaatacacaactactagtcctcttagaaggagaaagaaaaaaagaagaaagacttttctgcttggtttatttatcttctctcaggagttgaactctggaaattgaaactcacaccccctcttctaaattataatcatagttttgtaaaaagggcttaccttaactttgtagcaaatctgtactttatggattggcaaaaatgagctcaaataaataacccaatagcaacgtcctggtttatgctggtcggtggaagattccaaatttgttaggattctggaagcagaaaacagaatcaagcaaatcaagcggcatccagaggctttgctgttaaaaaaaaaaaattaagtgctctgggtagaaaaaataaagcccccggttagagcagagcaaacaaaaagaagaaaacaacgataaaaagaataaagaccaaaatgctctcccaaatcagagggaatgaagacactctctgggtggcatttgtgcaaggcatgaggctatgctggtggataaaaggccgggaagaagttgaaaatggttttagtttaactgtccagagccagagctgggtcctgggcggcgtggttttgagcaaggtcagtctttcattagctctcttgcacatcaagggaacgggcctctcacgtactcttctcgcctgagcaaagtttagatggcctagggtagaaatggcaagtaattaaagacagagtctatgggttttctgggatccttcgaaaacgccctcccaccccgcccgctattccgcagctccaccctagtgctttgtagccgcggcgctgggctctccttgcagctgcctctccttccagggcggctgcttgtcgagccaagtgggagtgaggcgtgctttttatagcagtcgggtgcaaagaggaagggggataaaaaggaaatcaagaatgaaaggaaaaagagaaaaagcggattacacggctgggcccggcggagatgtgtaatgtgaaacatcactggtgtcagctcggatatctcaggccaggcctctctccaatacacaaaagccgccgtctggggcgacagggaggcccgatgtggattgggatcggggttgcggctgggccaccggacacgggtggaagccggccggcctgggtggccgcctgcaaagccaaacgacccggctgggcctggcgcgcggacaggcctgtggtgggcttagggtaaagaagaggcagagcgaaagaagggggaatctccaaaactaccctttccgggttcccggagtttaatatgccaagctcctggagttaacgagctgacgaagaggtggtcttttgctctttatttggttgttttgctaggcgagaaagagtgttggcggcctagtccctgttaagggagcacgtaccagggggtgggggacgacagtggaggtcagggaaggaagggaggaattgcgtgggagaaagagcgatcctctagtgcccttccagccccttctcctcatccgtgggtctgtggctttggaatggaagcaagtttgcaaggtgccccgggaagggttggaaaagcctgctgccccgcgtttgttttacattaagtgtttttggacctggagaaacgcctggctgagtgatcaaaccgtccgcaggtctccatgcgttcggctgaggtttgtggcgtagctccgagtcccagctcgcaggccagagcagaccaggtctcctgcgcttggtggagacccgggccagtaactgaaagctggccctggtatcttggtgtgcagggcggtgcagtgaagcgaggccagggtgtgtgagtgcgctagcgtgtgtgtcgggggaaggcgggggttggcctccgatggaagttttagtaatctgcactgtggcatctgtttgctcccttgccccaaccgcccccaggtttggttcaagaatcgtcgggccaaatggagaaagagggagcgcaaccagcaggccgagctatgcaagaatggcttcgggccgcagttcaatgggctcatgcagccctacgacgacatgtacccaggctattcctacaacaactgggccgccaagggccttacatccgcctccctatccaccaagagcttccccttcttcaactctatgaacgtcaaccccctgtcatcacagagcatgttttccccacccaactctatctcgtccatgagcatgtcgtccagcatggtgccctcagcagtgacaggcgtcccgggctccagtctcaacagcctgaataacttgaacaacctgagtagcccgtcgctgaattccgcggtgccgacgcctgcctgtccttacgcgccgccgactcctccgtatgtttatagggacacgtgtaactcgagcctggccagcctgagactgaaagcaaagcagcactccagcttcggctacgccagcgtgcagaacccggcctccaacctgagtgcttgccagtatgcagtggaccggcccgtgtgagccgcacccacagcgccgggatcctaggaccttgccggatggggcaactccgcccttgaaagactgggaattatgctagaaggtcgtgggcactaaagaaagggagagaaagagaagctatatagagaaaaggaaaccactgaatcaaagagagagctcctttgatttcaaagggatgtcctcagtgtctgacatctttcactacaagtatttctaacagttgcaaggacacatacacaaacaaatgtttgactggatatgacattttaacattactataagcttgttattttttaagtttagcattgttaacatttaaatgactgaaaggatgtatatatatcgaaatgtcaaattaattttataaaagcagttgttagtaatatcacaacagtgtttttaaaggttaggctttaaaataaagcatgttatacagaagcgattaggatttttcgcttgcgagcaagggagtgtatatactaaatgccacactgtatgtttctaacatattattattattataaaaaatgtgtgaatatcagttttagaatagtttctctggtggatgcaatgatgtttctgaaactgctatgtacaacctaccctgtgtataacatttcgtacaatattattgttttacttttcagcaaatatgaaacaaatgtgttttatttcatgggagtaaaatatactgcatacaaattggtctggattctttctcccctcctctgtcactaacttggccaggacatctcagtcactgcttcctaaacaaaccagttccctctgcctgcctagttaaacatacaaggcagcagtccttatttaaatttggtagaaataaatgatagccattcatcagaaactaaaaagaaaaaaaaaggcattcccgggggggaaaagggctacaaaatctaattttgtctctccaatttttctttggcttaaacctagaggattccattatggctagcaaataatatgaaaaagaaaaaagaagaaagaaatttagcaagtccatcagcttaaaatgactctcaagtttactcctttacggggaaacctacacctctagcaaattgttctggagaaatatttgtgcatgtatacatgtatagtttatatgcatttctctcaggaggaatacatctataataaatttacagggaaacatctccagttcaaaatatttaggcttccacgtttatcttcaggcttaagtagagagatccttcatgttatattgcattactatttccaaatcctttggagacattaaaagaaacaaagatgatttctaataactacagcccttcagtttctcaaagaactcaggggttgagaggttagagtggagtttcctgagtcttgtcgagcaatatgtagttgaggcaaaggtcatgctcccggtgttttgttttaaataatattgacccattaattctaaacctgcttgttcctgaaattatacaggattatagtttgcaaactgcaggacaatgaagcaaatcaagatgaattacagccctggccctccctgccatcctctgacatctaaacagggaatgagttcggtgtgagtgtttaaatgaactttaagcacccgatccttctttatccgcgattttcagctttaaaaaaatgtgaaatttgatttcataacaaatagaaacaaataccacttagtcccagagaattcatcctcatggcgctaggagggtcgttgtggaggtggggggagggatgtgctgagatcttttgttatgcttgtcaaccccccgcacaaccaaagtgggcgagaacaaacaccacgctggggaacttagagcaaaaagtaaccgccgattttctggagccgacaatatcattgttttttcgccttagttgctttcatctgaatgaaactttacctagaagcttttggagctcgaacactgagtctttgttctgcaagaaactgctgctgccacttaaagagatcgcagataatgtccccgatttaagatcagtgctcaacgcacactttctttctttttaaagctctgcctccgacttgcggagggatcacgcagcagtggggggcagataaaagccctttggacgagggtcacctcccaccatgttgttcacttgagagcagtggagaggaaaacggtccccacgggcggactttggcttctggagccgcagggcccagcggtcccggcctctccgcctctcagcctggtagggggcggggagggccaaagggcggaggggaaggaaggagctagaagaggggatttggggatgggggctggaagcgctaacgagacctgcccggaggatttaggtctcctgcaggttggtgagtgatttgggagctctgggtaaaagaggtgtacctcggcctagtttggttgctgaactagaacaacgcgaggacatcaatcaatctgcaggaaacaaaaaatggaagtcgaggtttaggaagagttgtcacttccccgacttgagtggtgcaggctgggggcggagacttgggacctaagagaggccatttctctcacctcagctcccttcccttggacttccaaaaggaataattttactctctcctccgttctccccacaactctcatccccgctagcccgcaggccgcctgcccctttccgcacctccttttcccatccagcgagagaaacctagctcccagagccgg

SEQ ID NO: 2 - DNA, 22020 bp, Homo sapiens
ctcccttccctctccctgtgtctctccgcggatctctgaatctttctgtctttggttttctcattctcttccaacttttccatgagattgcctatcctcgccaccagctgaaggcaaggccgttctgctacgagcgcctcttaatctctacaaaatgaaaagaaaaaaagggaggattattagcccattactcagaggaatggggaggctgcaaaaatcgtcgatgggcagaggtgaagatgtctttctcggactgcactttccggtgtcctgtaactagagttcagttgtgggacttgttgaagaaatttgattttcttgcctcggcgagatttcaaaaaccagaaatagaaattctcagagtcagagaggaaatacaattaaacagcacgtgggcattttccccctcatttctctccccttaaataacactgctttgagtttccactgggtaaagagagaaagtttgagttttcacggatgttacgtggaggttagaaatggcttaaaatgtagatctctaatcagttttcttcgtggctgaagaggctaaccctttccataaaatgagtccatctgtcgactgttagctatttcaaagtgaagggatttagcactcaaaacaaattgagcaagtttgtttgcctgtttttactgctaactcaaatgaattcaaaacacggagtaattcaagaaaacacataacatgttccagacagcccccaaaagtagggaaagcccagcacctatatagtgactagggttagttttaagcgccaagcttttttaaacgtatctattttatgcacattctcccgagtcactatatatttctaaaattgcgagtattggtatattgatttaggaagagcaatacaacttttagagggaactttattctcaattagggaccaaagagatgtctttttaatagcgggcctgagttttgctctcaagcaggaattaatattggtgggaaaatccgaatccaggagcaatggctgtgttccggcactttccaaaaacatacattaacaggatgcccttgagattgaaaaaacattgtcccatatgcctggcagaagccttcacacctggtcctccaggcgaattatatttatagtccttccactcagaggcaggacagagccaaaatattctgctcactaccaaaatacacatttttgctcaagtcaagaaatcagaaaatcagggttcagaagtaaggcacacttttcgagtgagaatatgcccctgtaatttcacatactctttgctttgcaggagcaaatgtggacttgagggaaactctctcccccacccccacttctatcccgtgcaatgtaataccatcctcgtcaggaacctgaacctcgtcattttaagaaatgagatatccgtgactcagggtgaacttgttgaatgtaggtacagcagaggaaattctagactctatgagtgtttgagccttgtccagtgcaaacctttcgtgaacactgggtcagtgcgtggccgtgcccacctgtgcgcagacactctcagcatgcctggtccacccgccttgacatcgggcgcggtgtcccagctaagctgggcccagcgtcccggccttccccagctgacaagcgtaggtcgttcgctcccggcttgtggccctcccaccctctcccactagctcactccattgttgtagattctctctctcactcatcctctcccatccccaccgcgcccacctccactcccgccctctaccggtctctcactttcctccctccgcagtccctctttgctgtgacctctttcctcaactctgcaggcctgaaagaaggtcacacacgcacgctcacacccacactccacacgcctcgtcccaaacaaccccatgaacattgtcctttgttccgtctcttgggccactttccctgtcgttcctcccagcccgtcctgatttgctccccaaaagtacgtttctgtctccccgctgccctggcgctccccctttgatttattagggctgccgggttggcgcagattgctttttcttctcttccatcccatcctcccttctggtcctcctttccacagtgggagtccgtgctcctgctcctcggttggctcctaagtgccccgccaggtcccctctcctttcgctctcccggctccggctcccgactcttcggcccgctggcatctgcttccctcccctgcctcgtttctcgtcgcccctgctcgctccccccggcgctcgcccgggcgctgtgctcgctcctggatcgccagccgcgcagccgggctcggccggccgcccgcgcgccactgtgcagtggagtttggtggaatctctgctgacgtcacgtcactccccacacggagtaggagcagagggaagagagagggatgagagggagggagaggagagagagtgcgagaccgagcgagaaagctggagaggagcagaaagaaactgccagtggcggctagatttcggaggccccagtgcacccgtggactccttcggaacttggcaccctcaggagccctgcagtcctctcaggcccggctttcgggcgcttgccgtgcagccggaggctcggctcgctggaaatcgccccgggaagcagtgggacgcggagacagcagctctctcccggtagccggtaagtggaggccatctatcccgcagggatgtgagataatgcgagtctggaaatttgttccacttcggagaatcttcaccgtaggtgatttgtggcttttggggctaagtttcgcccaaggtaacgcagtcggcaaacagaccttgcaaagccctgttcctttcgtcccccgccacagacactaacaatctacagggtgctgaagtcgagagggaagccagaccgtggctggcatttaaaacgaggtatcttcccttaaatctcggtgccaacactgcaggaacaaatcctcgggccaaggattagcattctcaagataaagggctgggtacaaagtttcagctactggaagattagcccccttcccattgttatccattgggaaaaaaaagaaaagaaaaagattccatcttaactggcagttagtgacctctcaggcccaagcgaattacctgggagccaggcctggatgccaagctctcaccatttctttggattgtaactcctttaaattgatcaccagtcaactccaatctggcacttcaggagatacactttaaatggatgcagagaattattttccagctggagattaagaaaaaaattttcgattctaaacctccgaaatatgttcctcttttccagtttaaccactttactttcttaagcaatttagtaatcaaactatcataaggtggtgtgatttttttttactcttttgtgtgagtattgtcttactaaactaaacggaaaaaacttttaccattataaatgtaaatatcagaattcatacattctaaaatatttttatgaaaaattaatctgatttaaagaaatttccttgcatttgttttagtctatcaatcaaaactaaagatgcttttatcacacaaaatatcattttggcagaaatccatctaaaattcaaataccaataatatcaagaaaacaaagcacataagcaaaataaattgaagatttttgttgatgtaacatgagcatacaacatttcaataaccaaactttccctaaaaaattaaatagccacttcatttgtggaatgttttactttaactcagcaaaattacacttaaattatttaggtgctttgttcccttaagttaagcgtgtttgtcttcaaatgttccctaaagcacttatattaattggttgtaaagaacgcatacacatggtaaaatacagaactgaactgagcagtattttaatttcccttaaataattacttactacaaattaatttactggctaatttcacaatttagttcatttaaaacacatgttcctgtgctgtttatttttaaactttccattaaagattttgttatggggtaacaaagtgtatgaaaaggggggaaatgtgaaaggatctgggattattcgaactgtatttttcctgcactttcagtcttgcggtagtcatcagaaattattttttagcaaattgttttatttcttagggcttgcctgcctgctttgccatggttcctcatcctccattagccgtgtagtgctttttgtgtgctcacaatataaaacccaagttggccaaaacaagagtcccttggcatatacattccaactagaacatgaactttgggggtgagaactacctcccatcaggaaaagtctcccatctcaatttgtgagattagccattgaagccagttccgaagtctggcagccaaatttctcacagaagacttgtcttgatagggcaagtttaaggatcagcaggcgggaattggaggtctctttttaaaaaattatcttccccagttatttagactcagttcttctagtaggcctggtcattaaatgaagcataaaaatgcaagtctcaaggctcattttgactgcaaaataaatctccaagtcacaaggacatgtaggagtgagctaaggaacacgccttgaccttcttttcagtccttagagtggagctctatgagttcttgaagatttgttttgtattgctttgtttggtcttcagcactgaagcacggggaagtggggggaagcatgtgtaataattgactgactttacaccaagcaacgcaatctttttcttctgtatatttcattctttaaaaaaaataaataaataaaaactatttgcagttaccatctgcagtgctccggctaccagctaataatgcagccagttcagacatataaaaaaaaaagattatcgaaatgatgatgacatgcaaatttcctccgaaattatcataagtaaacatttgaagtctggactaataaaatcccatctgtgttacttcatatcgagttagtagaaagctgtgataatgaattttgtaatatctcacgaacagacatctcaatcagggactaatcctgtgattttactgcagaatcactaaatctggagccgccaaactgctacttctgggcccacgggcccacaaggatcgaatcggcagagtccccgcccgcgttctcgctagcgggtgggggaaccgcctggccgtccccaccctggatccccacgccacagcgccgggcagcccctcctgtaggcagcgaccttggccagaggctccccagggcccagctcccttcaggagaggccgagacgcagggaaacggtactcaggccagaggcaggcccgcagctccctgccccgcctctgtgcctccgccaacccgacaacgcttgctcccaccccgatccccgcacccgcgcgaagtgggccctccggtcgtcggcgtaccctggttagcgtggagagaggcaggcgctgagatcgaaggggcctagggagccctggaccttcttttcttgtctttaaagcaaccgcggctcttctacccacccggtggagcccctcgagacccacctctcccggcttgcctgtggcagagaagggggagcgcgttaaatgcttggctcgctgcgttgtggttgaaaacgtgaaaaagatttggctcgcccgggagagaaagggggagaactgggtagcagttataccagagctatttctccgtccttggcgggcagtaaaccctccaagaacgtttgccctgctctcctcagtctcgctcagtccacccagtgtctctcctctgcgatcttaaatcatactttagggtaattatttgtagtaagtaaataaatggccgggttagtatctctaggagaaagtgtggctaaatatggaaaagtggctcctgatggatgagaggcccgaactcagctcgctcctgaaacaccctaggccaagagcccgttcgtttcagaattacagaaaaccgagggaaactgctgtctaggacaggggcacgttggcgctgatgttctacaaatgtttaccgagctccaactaatggacaagcactgaagggtggtctttgcatacagcttcccaaagagaaaagtcctctccacccacccactcccgccgccattgcgttcagatgagttcttaaccccggcaccgagattcttgaaagtaggtccacagcctccccagcacactgtggctttatagtcctctaacctctgggcacttttgcggcaactctggagggagatcccctcttgataaataaatgtcctgggcccgaggctaggctggagatgctgctgcacagccagaggctgtcaggtcggaaaaacacgcctgaagcctagcacacagtaggcgcctaacagctagtgtaacgtagtctcatctgagccctgctcactcgacggccgccgcttcttacagccttccttctcttctgttttgcagataacggggaaatggagaccaactgccgcaaactggtgtcggcgtgtctgcaattaggtaagaacccccttctcctgcccgggtcatcggacgggaggccgcgccacgtgagggcggcaagagggcactggccctgcggcgaggccccagcgaggggcgcttccccgaggggccagcctgggcaggaaggaaatcagaaccaaatcgccagtggcctctccctgtggcggggcggtggactaggaagcagcggcgcgctgtgtaccgaagcccccagcctactcctcccggctggaaccgccggcaaccggggaggcgcagaaagagtacgccatcctgccccgggttgccagagggtccgggggacggggatgccgtcagctctttcctctaactgggtctttgctctttgtccctctttctcctccggcctgcctcgcccctccccctcctcccgtccccggctcctttcggccgcggtccgggacgcctctctccgcacgtggggcgggcgcgcgcgtggcccaggcgtgcagccggcggccgttgaatgtctcttctccaaagactccgaaatcaaaaaggtcgagttcacggactctcctgagagccgaaaagagcagccagcagcaagttcttcccgcggcagcatcctggcgccaatggtaaggccgggagggaagcgcaggccgcgcgctgggcacccgcctccgggactctgggcctcgggcgaaagcgcaagaaggcgaggccgccagacctgacgcgcctgctgcttgaacttagacactccgcctctgggtgggacgggaagcagccgtcccagggacgttaattcctttccccaaattatactgcactcctgagacccaacacctctctctccctctcgctcgctccctgtggtctgatcccctgcgcacgctccagcaaacctcggcgtctaggctggcgtggaaaagtggtccaacagcgacctctgtcgctcgttatatcccgctgcgcgcagcaccaccagggcccactcagtcactcaggctctcagccacgccccacccagacttgtgggtgcgcggttcatcgcgggaggcaagcaagggaaacttgagttggcgaaggtttgctttggctggttgggggaggggcggggggccgacaacatccctgaagagctggagggcagccactgtgcttagcagcccagggtagaatggaggtttgcccctgtcgacgcgaacctgcctgaagtactggttgccaggccttgggttttggcgatgtccttgcttgattggctggtttcactctggaggaatcgagggacattgccagaggaggtctacaggcttatgtaaaaagttaaaaagtctccaacctacgccacaggcctttcctgaattgaaacttgttctatggggcggaggggggggtgtaagggatgagagagggaaaatgcctttcttttcaaatacatggaaaaaaacccctcaaatttactgttcctctattttcctggctccgtagtaaataagtgcctagccttaggaggctattgacctttgataatgtgagcagataaagccccccccccccccccacagccctcggcccctaaccccctctttccggattaaagtgtaagaatacaaatgtaatatgggatggaggggggcgatttgggaccccggtcaaaaaaacaaaccgtattactaagaagaaaacaaaggtcttgcattggagtttccccgtgaatctgagagaaaatgatcatttgttgaaatgaagcgtctaaagcgatcctgtgcttcacgcccggacactgcactacaccgccagtcgccctgctgggccagttaaacgctcacttgtccgggattaaccccatggggttaaatgggggcaatgcagagataacgtcgcgtgatttctgtcatttagattgtgttaaattcttcttctgtttgataatcggtagtaaaaataaattattagaccgtagtatgtttgggatatggttaaaaatcaagagcagcgatgacttctggggagaatgctttgcgcgggctcagccctggttccggccagagtagaggagctccccaatctcgcttcgcgcggggcgggctcgcagtcgccaagccgaggctgacatttcttattgtgctgggagccagagagacgcaaaatgtctccttcccccagtccctaccccaggtttcctagacatggggaatgcattctgaggacaggtggagaagcctacggtaggatggggtcctcgtaggtgagcaggaaacggctaagagcagaggagttctgcttgcgccagtcacaagccgcgcaggcgcccctggtcgttcgcttttgataactagcacataaagaactagaaataatgaatgattgctttcttaaccattactttcaggcccgcattgttttagtgcacgtgaaaggctcttcccctacactcaatatgtctttttctactttttgaccgaaaagaaaaattgctgcctaaacacgtttaatgccattaattaagaaaaggcatgtaatgggaagaaatgctgaaaatcttgatttaattggcttttaaggaactagtagacgacaaaaaaaaatcacacgagtgggcaaagctatagcactgctgaaggatagagcacctatccttccctgattttaagttaacttatggaatatccaaagtcctggccacatcctgcttgtaaaacaaaaggatttatttcttgtgtttttttaaagtttttctttgcttttaaagagaaaaaaagttcacaatgacatatgacttcttcaaaaggccgtgatagtctattacgctaccctctccgcctctgcccccaacgccgcccaaaaataccatgtcctcgttaaagactaaacgttctgtataggcagagtccacctctaagcagtccaggctctgccttccttccctagtgagtcccattctccttggtatccattgggcggatgcccagcctggatagaaccccgaaacgggggtagcacgagagcgactggagacccctaaaagccagaggtttgagagagggtggacgcagctagcagaagatggtgtagaagccagctgagaacgaccccctagagcaaagagacctttctttggcttttcttgctttgggggtcctgaaaggaactcataaaatggtcttcaccctcaggaggaggacggactgaccctcctctgtcactggcttaaaaagtttcagggcggcggctctgggttccgtgctgaaatcggactgcactgcagcccctttggacctgacgcctggctttgcgtcccgacaaggggcgggtacttcccccggcctcccccaggaacgcattaattgttaaatagctttggcccagtggatgggctgaaagtgttcgacccaagtcgctggtgtgcacagacttcctccctctgggaggtgggctccatggcccgttgtggcacccccagccgcgacacacaccttcacacgcggcagcagctcggtcccaaccttcttcgaaggacctgggttaaccctggcggccttggcggccgcagacccctctccgccgccccgcccccgcgcctctcattcaatcagcgaatgtttgcggagcatatgctacgtggactcctaatgtaccccctgaaagcaaataatacagttttcctcgccgtcatgaagggattttaatcctaacatggacaccagcgagatcagcccagaccgtctctagcaaaacgcaaaatggtggtgcgtggggtggtgaccaaggtcctgagccttgtcagaaagaaggggatgtgtagagaaaggtggagaactctagctgtggctagcgcggaagggacaggtgcttgccgaagggggcatgaggcttgaggaaaaagcaacgaaataggggtaaggagagttttccattttctttcctccgcccgacctccgccatcccattctcccctcccctcacccaccccccgcgccaatcaaatctgcagctcacttgaaaggtgccccgccgcgttgtggtttttcacccccaggggaaattgtaccagttgtccgaaagtagtcagtccctgcggatccctgcccgcaaaagtggtcttcacaggtcgcgttcctcgctgctgactcggtacacaaagtttcttaaggctggttcggctgttattcctcaccgcccgctgctaatatatgcagcagttgttagagcggctcgggggaaaaggaaatgtataacgaaagcttattcgtgagcaggaatatactaatggaacaatctgatgtcttcttaatcctatgtaaaaagctttgtcgtcttcctaatattgactgaatgggtaattaatggctcttcatctacgcgaataccctgtaatccaagataggcaaaagactacaagcccaaggtaagaagacaaaaggcccaactgcagcggcgtttgcccgcctcctaacccccagggtctctgactatgagagtcttctctcatacgagaaaaagcaggagtgggagaatatatacctatcatttcggggctagacttcaccgcagcacctgaccacccagctcagtcttctgttactctgtctttccacctccagtccgtctccctgcattttttttcttcttaactcctctaggatgacctcctaccaccaccgccattatggtcctaataacctttccccctaaaccccacatctttcacttcagcaaccaatgaggctgttttctgatccaggaggagattcttttcttttagaaccccatgcgtacagtctttgagaactaaagtagttggtaggggaggaagaaattaatagaaagggagagagcatacagaattgtgtgtgtatgttaaagagcgaccaggaatgacagagtccacttctttgtgaggatctgacgggaagagtgtccaacactctacagcatgtttttaacaggctgacattttaatttaaacctttagaagtaatatattacttgggttactacaatgaggtgggttcctttttttttgtcagctgacagcttttaaaatattatttcgctagggaaataaaagcttcatctcagaattataggtgggtatttttggatttaggtgatttatggtcaccatgacaactaatgctgaatgttagctaccaacatgtctgggagagagaaaacagaaagaagggagagcaaaagaaatagaaaagggagatggacataacctggagagggaagaaaagagaaaaagaggaggacagatgagtgtttaatccaactgtggtttaaaaaagtggcgggggggtgggatttcatttaccccctagcaatttctttctttcaggacgtggatatatatgttaaatttaatattcaacctttccttccctttctcccaaacacatgtgctataccattcttccttatttaacttagttcggcaagtagttgctttctggagactcagtgacacctaggaaaactgtggcagtaacatgcaaatgtgaggaagcattaacagcatgttcgttgagtgattttagcaaatgccccctcctctaatctctttctcttctcttccccaggccactgtgaggtggtaactcccactgccatctgaacactgttcccccaggtagttaccccaaatctcaaatggttgagcagctagagctgtgggttggaaaaatgggtaccatttgcagggacccagagagggtggctgttgctcaatatatctacagattccaattcagaaaataatcttccccttttgacaagccagagctttttaaattccatttaggaaatggggaaaaggacagctacagtgaagcttttaatttctgggttatttggttccatagctatgaggggtggtgggtagtggactgttttcagcttggtttgtatgcagagaaaagccagatattggagggggtggggcacttctcgggcaggatgcaaggtctccatctgacctctgcgccttaccaggagctcacacactcacgcccatcacgtggctccaagctgagtccaggcgggcctcgtccttgagctagcttgggcagggcaggactcccacccgtctaaggcctaacagctcagggagatgtctaattaagccatcttctgggtgaactctgaagacagactctttccaaaagtcagagatcacttggttgagtcccaggccagattgatacggagagtttggcggcacagcccaaccgctcactgccatggccagagggactttgcacaactaacactgaagagtgtgaaattaaataagactttaagactggtaatcggtggcaaataccagcacaaaacacggctgaccccatgtcacacatttccctcctctagggtctttctttgaaagaataagcaagaaaccccaatcgagacaacccctgatgtctcccagactcaaaacctcacgccggcactgggctttccttctttgccccacgtgagccatgcagcacctctagtttccctaccaggatcccaccaatgttctccgtattggaaatttctgtgttagaggctgaactcatagtaatttctaaaaccaattaagaagaacttagccagaggtcacagtaatgctggaatcacaaaatgcataagatttattttcttctggcccctttctcatccatgctgcctatgcctgtgcacccacaagtcttatgtacattaaatctccaaaatcaaccaccaccatgccacagagttttcactggacagtgtttcttagttcccaccacatacctccttttccccatgcagacttatcatgttggtgtcctgccacacagggggcctgagaagaatgtcacccaatcgccgctgctgtgagtgtgtaaagtgattaggagattaggagaatgttgaaactcctgctggaaaaatgcaaagaaaatcctcactttgagtcagttgtttacagagccagtgtgtgtgcgtgtgtgtgtctgtaatataaaatggatgtgaatatatatatacaaatagatatggttttgcttttactttaatctgaattatctagataattgtctttattcaccacttgattcaatggtccacacaaattaggacaccttatctttttaggcatctaggctgctgctgatccccagtgcttccaacatctcgcacacgttggcactatgaggagcagtcacgtgcccttgggtttttcaaccactttggaggctgattgaggtccttcatacatgtatatttgtcgtgatgaaagttccatcggtagagtggagccaccagagcttttatcaaaattctgtgggtttatgagagatgggtttagaaatctatatggctctgtggggcttctcggctttctaaaataaggcattaacacccaagcttccaaaaatatttgcagctctggggtttgaatcttgaaaaacaaggagtgaggggctgtgtatactaactacagtggagatttttttcattctttaatgtgatggagtcccttcatgaaatgaagctttaaggggcatggtattgtggggaccacagctattctgaggtttaaaagaagaaactggaatatgattagtaaacacattcagcagaaaagagctggattcttattgacttagttataggtcatcggctggcagtgcaatgggaggaaatatttactttacacatatactttatgatcttgggggaattagaggaaattcaataagaaaacggctagaaacatttaaaacccttatttaaaagacttaagcaaattagagtcttatcagattaaaaaccactacaaatgtaagagcattgtcttcagtgaaacgctgtggggtctgagaaggagattctccgccaaatctccgggataaaatgcgtcatttaagcaccagataatgagcagaatgtaaattaatttaaccttctttaccaacaggctgctagtgtaatgtgtataatttagtgataagattgcaggacctaatatagctggatgtatgagcctcagctaatgcagacctgtcacatgaggatgtgttttactctgagcaggtgtctgtatgtgtggaatggggtaaagtggaataaaaggttaaaagcagaaatgctgatttaaagcttactatgaagaaattcctcccttgcagctaaattattcttaaagtgggatgatactggtgaagaaagactgaaaaacaattctcatgtgcgtctttggactgcaagtttaaaatggggaggagttgcagatagggtttgggggtggtcagggcaaaggagagacacataagttgcaaatatatttgtagtctgcttcatccactttgttccacatcgaataagtttcccaatcttgtgaacaaggacaaggagggagtgttttaaagatacttcatgctggcattgcaaatcattgactgtaatgtcaaacaaatacacattcagagatgataacactaactccatagtaaaacaatcgcccatgcagaaacccagaggagattagtttgtcctctccagctgacctatgctgggggacaaaaggactttcaaaaattattttgaatatgtttggatttctttctttaatttctttggaaattaaatttgcttggaaacagtgctataaagagttgatgtctccaaaggtgattttttttgttttatataaataaggttttgcttttgctagttgagcgcagttctaggcttttcgcccttagctcacacacaccccttctgcctgcttggactttaatggctcaagacagccttgagctcactgggaaaagaaaatgactgttaaaaattatccttgaaattggttatttggcaacattcttaattgtatggaaattcattaaggcatatttcatatataattagctcaaggttgttgattctacaggctttatggatttaaatctgattgataataaagtaaacaagagagtcgaatttaaagcgtggctctctcgggttaggacgagcttaatacagtgtacaaggaatttgaaagatctaggatatgtgtcttaatcaacgttaagtagaatggataagctttcagcattttgaaaacgctgggttagggtttctcttctattgtgtgttttctgtctggggactaataagcatcacagagaacgtgatctgaggcgactttttattcttgtataaatccagagtgaaccaccaaacagttgttcgtttaaagtcaaggtaattttcttttgacgggtccatttgcttctcgatttctaatttattagcctgccttttcagggctctgtcttctttgcaattaaagcttcttcagattagcgcagcattcacttgacaggctgtttggaaaatttaagatcggagaggtgatttgttgctgtttttcaaattttctagttttaagtaacgtgtctcctttttatatggggtgggggattggaaatggatgtagtgagacacaaagagtgggtgtcttgttgatccttgtacctttctcttcttgaccattccactctcttctcccaagccttcgactcctagcctcatctcttcacctttgggttcgtactaaaagccggatcgccttgggctgggcaggagctgaattcccgggagcttgcctgtgtagacccagtgcgcacggcgaggcagtagcccggccccgcactgctgataggtgcaggcaggacagtccctccaccgcggctcggggcgtcctgattggtgcggagccacgtcagtcgcacccggagaagggtctgggaggaggcggaggcggagagggctggggagggccgcggcggagtgacgtctcggcaccaggaagcccgcctctggttttaagatgttaggccaacagggaagcgcggagccgcagatctggtccgtcgctcgcctgggtgcctggagctgagctgcggcaaggcccggctcctgttcgaccgcccgaggggtgtgcgtgtgcgcgttgcggagggtgcgctcagagggccgcgtcgtggctgcagcggctgctgccgccgcaggggatctaatatcacctacctgtccctgtcactcttgacacttctctgtcagggctgccgcgtgggggggggcgggcagagcgcggtcggcgttagctttccttattggaggggttcttgggggagggagggagagaagaagggggtctttgcccactcttgtttcgctttggagcttggaagcctgctccctaaagacgctctgagtggtgcccttctgcccacatcccatgtcttcgtttgcccgctgactttccgtctccggactttttcgcttgagccttccggaggagacgggggcagcttggcttgagaactcggcgggggttgcgtcccctggctctccccgcagcggggaaactccgcgcctagagcgcgacccggagcgggcagcggcggctacgggggctcggcggggcagtagccaaggactagtagagcgtcgcgctccctcgtccatgaactgcatgaaaggcccgcttcacttggagcaccgagcagcggggaccaagctgtcggccgtctcctcatcttcctgtcaccatccccagccgttagccatggcttcggttctggctccggtcagccccggtcgctggactcctccaagcacaggctggaggtgcacaccatctccgacacctccagcccggaggccgcaggtaaggcgccgcgccgccctgcagacattcccgcttagctgctctgcgccacccgctccctctcgccccaaggaagtcagcccctccgcggggaggcgtggtgggagtggtcgttcgcctggctccccgcagaacttccgggagccggaattttgactaccccgcatccctttagttctccctcgaccggcccggctcctggggcgctaagggcgcgagcaattctgccgccctctctattcgtaccctggcctcccttctgtttcctgggtcacaaaaatcccagcatcttgattcgaggaccttcagaggccgccgacctctgtccctgttttcctctcggctttcagctcccgaggagctccactcgttaggaaattgcctgaaaccactcagaaatgcccttcgcgaagaggcatttttttttttttttgggaaagggccggcgaacttcggtgcccaaccgaatccccacatcttttcctagccttcccaaaccgcatggaaatctgagctttctgcgagggggaggggggtctgtaaaccacgcgcgtgtgcgcgtcccaggagatttggtgtgtctgcgcagaggtgtataaatatacttgaaagcacaggctataaaagtgaatgtgccgctgcagtgagataaacatgtaaataaaacgtgcggcgctgggggaggggaggaaatggggcgcggacacccacacttgcgacctgcacaccccacaggcgcagcgctcctcgcggcccggagccgccgcgcgcaccctcctccggcgccaggcagcccagctcttccacggcttctgccgccggtccagttggcgtccgcgttgcaggtgggcatgctgacgggaaagtgtgtgtgtttcgttttcagagaaagataaaagccagcaggggaagaatgaggacgtgggcgccgaggacccgtctaagaagaagcggcaaaggcggcagcggactcactttaccagccagcagctccaggagctggaggccactttccagaggaaccgctacccggacatgtccacacgcgaagaaatcgctgtgtggaccaaccttacggaagcccgagtccgggtaggagccagcacggagtctgggagggatggggggaggatgttgtggaggtacaggccaagtagaccaggagagaatgtggaaggcagcgccgcctgggagggcgccggtggggcgcagctttgcaaaggcagaaggcctcgcggcggcctggttgcgagattacagttccctctccgaggccgacaggactgccgccctggctcaggctcccagagcggcaccggctcactgccccgccatcccgcgatctcacgagctgggctgcatgggcaatcccctgcacaggacattgtgttcctggcttgcagttgccagagcagagctaataaaatccctaccaggccaagagccgcgaacaggctccaacctgtgagcctttaacaaggaaaacccgccagagacacggaagagttggccctccctgggaaacctttgtcccggccctggcccagctttttccctcctgggctcgcgcttcttacaccttctttacggttgtttcggccattcaggtctctcccacacaccctatttcctagttttgtgatctccgggagcaaagttttaatacacaactactagtcctcttagaaggagaaagaaaaaaagaagaaagacttttctgcttggtttatttatcttctctcaggagttgaactctggaaattgaaactcacaccccctcttctaaattataatcatagttttgtaaaaagggcttaccttaactttgtagcaaatctgtactttatggattggcaaaaatgagctcaaataaataacccaatagcaacgtcctggtttatgctggtcggtggaagattccaaatttgttaggattctggaagcagaaaacagaatcaagcaaatcaagcggcatccagaggctttgctgttaaaaaaaaaaaattaagtgctctgggtagaaaaaataaagcccccggttagagcagagcacactaaaagaagaaaaccacgattcttcgaataatgaccaaaatgctctcccctatcagagggaatgaagacactctctgggtggcatttgtgcaacgcatgacgctatgctggtggataaaaggccgggaagaagttgaaaatggttttagtttaactgtccacagccagacctgggtcctgggcggcgtggttttgatcaaggtcagtctttcattacctctcttgcacatcaagggaacgggcctctcacgtactcttctcgcctgagcaaagtttagatggcctagggtagaaatggcaagtaattaaagacagagtctatgggttttctgggatccttcgaacaacgccctcccaccccgcccgctattccgcagctccaccatagtgatttgtagccgcggcgctgggctctccttgcagctgcctctccttccagggcggctgcttgtcgagccaagtgggagtgaggcgtgctttttatagcagtcgggtgcaaagaggaagggggataaaaaggaaatcaagaatgaaaggaaaaagagaaaaagcggattacacggtctgggcccggcggagatgtgtaatgtgaaacatcactggtgtcagctcggatatctcaggccaggcctctctccaatacacaaaagccgccgtctggggcgacagggaggcccgatgtggattgggatcggggttgcggctgggccaccggacacgggtggaagccggccggcgtgggtggccgcctgcaaagccaaacgacccggctgggcctggcgcgcggacaggcctgtggtgggcttagggtaaagaagaggcagagcgaaagaagggggaatctccaaaactaccctttccgggttcccggagtttaatatgccaagctcctggagttaacgagctgacgaagaggtggtcttttgctctttatttggttgttttgctaggcgagaaagagtgttggcggcctagtccctgttaagggagcacgtaccagggggtgggggacgacagtggaggtcagggaaggaagggaggaattgcgtgggagaaagagcgatcctctagtgcccttccagccccttctcctcatccgtgggtctgtggctttggaatggaagcaagtttgcaaggtgccccgggaagggttggaaaagcctgctgccccgcgtttgttttacattaagtgtttttggacctggagaaacgcctggctgagtgatcaaaccgtccgcaggtctccatgcgttcggctgaggtttgtggcgtagctccgagtcccagctcgcaggccagagcagaccaggtctcctgcgcttggtggagacccgggccagtaactgaaagctggccctggtatcttggtgtgcagggcggtgcagtgaagcgaggccagggtgtgtgagtgcgctagcgtgtgtgtcgggggaaggcgggggttggcctccgatggaagttttagtaatctgcactgtggcatctgtttgctcccttgccccaaccgcccccaggtttggttcaagaatcgtcgggccaaatggagaaagagggagcgcaaccagcaggccgagctatgcaagaatggcttcgggccgcagttcaatgggctcatgcagccctacgacgacatgtacccaggctattcctacaacaactgggccgccaagggccttacatccgcctccctatccaccaagagcttccccttcttcaactctatgaacgtcaaccccctgtcatcacagagcatgttttccccacccaactctatctcgtccatgagcatgtcgtccagcatggtgccctcagcagtgacaggcgtcccgggctccagtctcaacagcctgaataacttgaacaacctgagtagcccgtcgctgaattccgcggtgccgacgcctgcctgtccttacgcgccgccgactcctccgtatgtttatagggacacgtgtaactcgagcctggccagcctgagactgaaagcaaagcagcactccagcttcggctacgccagcgtgcagaaaccggcctccaacctgagtgcttgccagtatgcagtggaccggcccgtgtgagccgcacccacagcgccgggatcctaggaccttgccggatggggcaactccgcccttgaaagactgggaattatgctagaaggtcgtgggcactaaagaaagggagagaaagagaagctatatagagaaaaggaaaccactgaatcaaagagagagctcctttgatttcaaagggatgtcctcagtgtctgacatctttcactacaagtatttctaacagttgcaaggacacatacacaaacaaatgttttgactggatatgacattttaacattactataagcttgttattttttaagtttagcattgttaacatttaaatgactgaaaggatgtatatatatcgaaatgtcaaattaattttataaaagcagttgttagtaatatcacaacagtgtttttaaaggttaggctttaaaataaagcatgttatacagaagcgattaggatttttcgcttgcgagcaagggagtgtatatactaaatgccacactgtatgtttctaacatattattattattataaaaaatgtgtgaatatcagttttagaatagtttgtgtggtggatgcaatgatgtttctgaaactgctatgtacaacctaccctgtgtataacatttcgtacaatattattgttttacttttcagcaaatatgaaacaaatgtgttttattttcatgggagtaaaatatactgcatac

SEQ ID NO: 3 - DNA, 144 bp, Artificial Sequence
  Bisulfite-converted PITX 2 sequence with a fully methylated status to which sequence a Taqman probe for methylated sequences will bind
gtaggggagggaagtagatgttagcgggtcgaagagtcgggagtcggagtcgggagagcgaaaggagaggggatttggcggggtatttaggagttaatcgaggagtaggagtacggatttttattgtggaaaggaggattagaa

SEQ ID NO: 4 - DNA, 144 bp, Artificial Sequence
  Bisulfite-converted PITX 2 sequence with an unmethylated status to which sequence a Taqman probe for unmethylated sequences will bind
gtaggggagggaagtagatgttagtgggttgaagagttgggagttggagttgggagagtgaaaggagaggggatttggtggggtatttaggagttaattgaggagtaggagtatggatttttattgtggaaaggaggattagaa

SEQ ID NO: 5 - DNA, 144 bp, Homo sapiens
gcaggggagggaagcagatgccagcgggccgaagagtcgggagccggagccgggagagcgaaaggagaggggacctggcggggcacttaggagccaaccgaggagcaggagcacggactcccactgtggaaaggaggaccagaa

SEQ ID NO: 6 - DNA, 24 bp, Artificial Sequence
  PITX2 reverse primer (PITX2-R)
ttctaatcctcctttccacaataa

SEQ ID NO: 7 - DNA, 22 bp, Artificial Sequence
  PITX2 forward primer (PITX2-F)
gtaggggagggaagtagatgtt

SEQ ID NO: 8 - DNA, 20 bp, Artificial Sequence
  PITX2 probe for methylated sequences (PITX2-Pm)
agtcggagtcgggagagcga

SEQ ID NO: 9 - DNA, 27 bp, Artificial Sequence
  PITX2 probe for unmethylated sequences (PITX2-Pu)
agttggagttgggagagtgaaaggaga

(19) United States
(12) Patent Application Publication
(10) Pub. No.: US 2020/0181712 A1
(43) Pub. Date: Jun. 11, 2020

(54) A METHOD FOR PREDICTING A BREAST CANCER PATIENT'S RESPONSE TO ANTHRACYCLINE TREATMENT

(71) Applicants: Klinikum rechts der Isar der Technischen Universität München, München (DE); Helmholtz Zentrum München Deutsches Forschungszentrum für Gesundheit und Umwelt (GmbH), Neuherberg (DE)

(72) Inventors: Manfred Schmitt, München (DE); Rudolf Napieralski, München (DE); Elisabeth Schüren, Tutzing (DE); Michaela Aubele, Kirchseeon (DE); Magdalena Absmaier, München (DE); Eva Gross, Germering (DE); Tibor Schuster, Montreal (CA); Heinz Höfler, München (DE); Marion Kiechle, München (DE)

(21) Appl. No.: 16/302,848

(22) PCT Filed: May 19, 2017
(86) PCT No.: PCT/EP2017/062192

§ 371 Date: Nov. 19, 2018

(30) Foreign Application Priority Data
May 20, 2016 (EP) 16170625.4

(51) Int. Cl. C12Q 1/6886; C12Q 1/6806

(52) U.S. Cl. CPC C12Q 1/6886; C12Q 2600/106; C12Q 2600/154; C12Q 1/6806

(57) ABSTRACT

The present invention relates to a method for predicting a breast cancer patient's response to anthracycline treatment.

### CROSS REFERENCE TO RELATED APPLICATIONS

This application is a National Stage Application of International Application Number PCT/EP2017/062192, filed May 19, 2017; which claims priority to European Patent Application No. 16170625.4, filed May 20, 2016.

The Sequence Listing for this application is labeled “SeqList-29Sep18-ST25.txt”, which was created on Sep. 29, 2018 and is 65 KB. The entire content is incorporated herein by reference in its entirety.

### FIELD OF INVENTION

The present invention relates to a method for predicting a breast cancer patient's response to anthracycline treatment.

### BACKGROUND OF THE INVENTION

In Germany, presently there are 75,000 diagnosed cases of breast cancer. In the year 2012, globally, 1.7 million cases of breast cancer have been diagnosed. According to estimates of the International Agency for Research and Cancer of the World Health Organization, incidents of breast cancer have increased by 20% since 2008, and mortality has increased by 14%. Prognosis factors provide information about the probability of the further course of the disease (disease recurrence, absence of disease recurrence and overall survival), whereas prediction factors provide information about the likelihood of a patient responding to a specific therapy. Clinically used histomorphological prognostic factors comprise the of axillary lymph node status, tumor size and histological degree of differentiation as well as invasion into lymph or blood vessels, tumor staging, various molecular markers such as the estrogen receptor (ER), progesterone receptor (PR), urokinase-type plasminogen activator (uPA), plasminogen activator inhibitor type 1 (PAI-1), and prognostic multigene marker panels such as Oncotype OX®, Mammaprint® or the Endopredict Test®. Currently, there is no test kit available commercially which would provide information about the responsiveness of a patient with respect to specific chemotherapeutic agents.

Approximately 15% of breast cancers can be classified as triple-negative breast cancer (TNBC). These are known for their poor outcome, early disease onset, and high aggressiveness.[5, 6] TNBC is characterised by the lack of estrogen receptor (ER), progesterone receptor (PgR) and absence of amplification of human epidermal growth factor receptor 2 (HER2).[10, 40] Consequently, patients suffering from TNBC do not derive benefit from established therapies targeting these receptors. Despite its definition, gene expression profiling revealed that TNBC constitutes a very heterogeneous disease in need for further classification, with overlapping characteristics with e.g. basal-like breast cancer and claudin-low intrinsic subtype.[28, 29, 33] At present, chemotherapy remains one of the most common therapeutic approaches in TNBC and both in neoadjuvant and adjuvant settings, anthracycline- and taxane-based regimens are most frequently applied.[38] They can lead to severe side-effects though as e.g. cytotoxicity of anthracyclines can cause cardiotoxicity and secondary leukemia.[16] Furthermore, an ambiguity concerning the optimal chemotherapy regimen for TNBC remains as contradictory results regarding the benefit derived from anthracycline treatment were revealed.[27] Due to the poor outcome and unavailability of targeted therapies, further characterisation and classification of TNBC according to prognostic and/or predictive markers could improve the currently poor outcome of affected patients.

Relatively new approaches involve the field of epigenetics. DNA methylation plays an important role in controlling gene activity and nucleus architecture, generally promoting chromatin condensation and transcriptional inactivity.[15] In breast cancer, genome-wide DNA methylation profiling assays revealed about 2,000 hypermethylation and 1,500 hypomethylation events [26], including several genes regulating cell invasion, cell-cycle, apoptosis, DNA repair and metastasis.[15]

Due to the severity of TNBC and due to the severe side-effects of currently applied chemotherapy, it would be desirable to have a methodology at hand which allows to predict the likelihood of success of a chemotherapy in a TNBC patient. More specifically, it was an object of the present invention to provide for a methodology that allows to predict a breast cancer patient's responsiveness to chemotherapy, in particular anthracycline chemotherapy. More specifically, it was an object of the present invention to provide for a methodology allowing the prediction of a TNBC patient's responsiveness to anthracycline treatment.

### BRIEF SUMMARY OF THE INVENTION

### All these objects are solved by a method for predicting a triple-negative breast cancer (TNBC) patient's response to anthracycline-containing polychemotherapy, said method comprising:

i) contacting genomic DNA isolated from a biological sample of said TNBC patient with at least one reagent, or series of reagents, that distinguishes between methylated and non-methylated CpG dinucleotides;

ii) determining, based on such contacting of i), a methylation state of at least one CpG dinucleotide sequence of the paired-like homeodomain transcription factor 2 (PITX2) gene and/or of one or several regulatory sequences thereof within said biological sample, and

iii) making a prediction of said TNBC patient's response to anthracycline-containing polychemotherapy based on the determined methylation state.

In one embodiment, said polychemotherapy is adjuvant polychemotherapy.

### BRIEF DESCRIPTION OF THE SEQUENCES

Furthermore, reference is made to the following exemplary sequences wherein the SEQ ID NOs: denote the following:

SEQ ID NO: 1 and 2: Two exemplary forms of the PITX2 gene which can also be found under Worldwide Website: ncbi.nlm.nih.gov/nuccore/161086966 or NG_007120.1 or Worldwide Website: ncbi.nlm.nih.qov/nucleotide?cmd=Retrieve&dopt=GenBank&list uids=13183092 GenBank: AF238048.1

SEQ ID NO:3: An embodiment of a partial PITX2 sequence that has been bisulfite-treated and that has a fully methylated status to which probes for methylated sequences will bind; this is one of the specifically preferred sequences in accordance with embodiments of the present invention.

SEQ ID NO:4: An embodiment of a partial PITX2 sequence that has been bisulfite treated and that is unmethylated; a probe for unmethylated sequences will bind to this SEQ ID NO:4.

SEQ ID NO:5: This is the genomic bisulfite-untreated sequence of SEQ ID NO: 3 and 4.

SEQ ID NO:6: This is a PITX2-specific reverse primer for amplification of a partial sequence of the PITX2-gene, in this case of SEQ ID NO: 3 and 4.

SEQ ID NO:7: This is the forward primer for amplification of SEQ ID NO: 3 and 4.

SEQ ID NO:8: This is a PITX2-specific probe for methylated PITX2.

SEQ ID NO:9: This is a PITX2-specific probe for unmethylated PITX2.

### BRIEF DESCRIPTION OF THE TABLES

Furthermore, reference is made to the tables, wherein,

Table 1 shows the adjuvant chemotherapy regimens employed in the patient collective used in the Examples

In the tables, the letters mean the following:

C: Cyclophosphamide

M: Methotrexate

F: 5-Fluoro-Uracile

E: Epirubicine

Example: CMF: Polychemotherapy containing Cyclophosphamide, Methotrexate, 5-FU+sequential Chemotherapy with consecutive poly-chemotherapy regimens.

Table 2 shows the patients' clinical and histomorphological characteristics. TNBC means triple-negative breast cancer; ABCS means anthracycline-based chemotherapy subgroup; NABCS means non-anthracycline based chemotherapy subgroup; NCS means no chemotherapy subgroup.

Table 3 shows results of the uni- and multivariable Cox regression analyses of the ABCS-group, i.e. the anthracycline-based chemotherapy subgroup.

Table 4 shows the specifications concerning the PITX2 probe and primer system. Primer and probe sequences:

PITX2-R
(SEQ ID NO: 6)
ttctaatcctcctttccacaataa
PITX2-F
(SEQ ID NO: 7)
gtaggggagggaagtagatgtt
PITX2-Pm
(SEQ ID NO: 8)
agtcggagtcgggagagcga
(containing 3 CpGs)
PITX2-Pu
(SEQ ID NO: 9)
agttggagttgggagagtgaaaggaga
(containing 3 CpGs)

Table 5 shows the layout plan of preliminary test data for qPCR robustness assessment.

Table 6 shows the statistical results of a dilution series of MCF-7, tissue X and tissue Y and the corresponding mean values and coefficients of variation. Tissue X and tissue Y are just randomly selected cancer tissues from two patients, from which DNA was isolated, bisulfite-converted and then separately assessed by qPCR in standard dilution series of these two samples.

Table 7 shows the statistical results of a positive control, negative control, cell line MCF-7 and cell line MDA-MB 231 and the corresponding mean values and coefficients of variation.

Table 8 shows the findings of various previous studies which examined the methylation of PITX2 in non-TNBC patients.

### BRIEF DESCRIPTION OF THE FIGURES

Furthermore, reference is made to the figures, wherein

FIG. 1 shows the Kaplan-Meier analyses of primary end points using a PITX2 DNA methylation cut-off value of 6.35. More specifically, FIG. 1a shows the disease-free survival (DFS) by PITX2 DNA methylation; FIG. 1b shows the metastasis-free survival (MFS) by PITX2 DNA methylation, and FIG. 1c shows the overall survival (OS) by PITX2 DNA. According to FIG. 1, the anthracycline-based chemotherapy subgroup (ABCS) was subdivided by the cut-off value of 6.35 into a subgroup with low PITX2 methylation (≤6.35, n=18) and into a subgroup with high PITX2 DNA methylation (>6.35, n=38). Patients with low methylated PITX2-gene had a significantly worse DFS (p<0,001, see FIG. 1a), worse MFS (p=0.006, see FIG. 1b) and worse OS (p=0,005, see FIG. 1c) than patients with high methylated PITX2.

FIG. 2a-c shows the assessment of qPCR robustness of the PITX2 marker assay. More specifically, for these experiments, two different PITX2 DNA methylation measurements were carried out per each sample. On the x-axis, the hatched column depicts the first measurement/qPCR run and the solid column depicts the second measurement/qPCR run. The determined PIXT2 DNA methylation scores are depicted on the y-axis.

FIG. 3 shows the linear regression analysis for independent assay replicates of TNBC samples. More specifically, for each of the 10 samples, two DNA methylation measurements were carried out. The x-axis depicts the PITX2 DNA methylation score of the first qPCR run and the y-axis the PITX2 DNA methylation score of the second qPCR run. Linear regression analysis resulted in an R-value of 0.93.

FIG. 4a-c shows a stepwise Cox regression analysis for multivariable biomarker analysis in the anthracycline-based chemotherapy subgroup (ABCS). More specifically, on the y-axis, the different combinations of the variables are pictured with M=PITX2 DNA methylation subgroup (≤6.35 vs.>6.35), A=age, G=grading (G1/2 vs. G3), T=tumor size (≤2 vs.>2 cm) and N=nodal status (positive vs. negative). The x-axis depicts the different calculated hazard ratios and the respective 95% confidence intervals.

Furthermore, reference is made to the examples which are given to illustrate, not to limit the present invention.

### DETAILED DESCRIPTION

In one embodiment, said prediction of said TNBC patient's response to anthracycline-containing polychemotherapy is based on whether the determined methylation state of said PITX2 gene and/or of one or several regulatory sequences thereof exceeds a defined threshold, wherein said prediction is made in terms of one or several parameters selected from disease-free survival (DFS), metastasis-free survival (MFS) and overall survival (OS), respectively, and wherein said prediction is negative, if the determined methylation state is equal to or does not exceed said threshold, and wherein said prediction is positive, if said determined methylation state does exceed said threshold.

In one embodiment, said contacting in step i) comprises contacting genomic DNA isolated from said biological sample obtained from said patient with at least one reagent, or series of reagents that distinguishes between methylated and non-methylated CpG dinucleotides within at least one target region of the genomic DNA, wherein the target region comprises or hybridizes under stringent conditions to a sequence of at least 16 contiguous nucleotides of the PITX2 gene and/or of regulatory sequences thereof, wherein said contiguous nucleotides comprise at least one CpG dinucleotide sequence.

In one embodiment, said one or more reagents comprise bisulfite, hydrogen sulfite, disulfite or combinations thereof.

In one embodiment, the step of determining the methylation state is performed by oligonucleotide hybridization analysis, methylation-sensitive single-nucleotide primer extension (Ms-SNuPE), sequencing, quantitative real time PCR or oligonucleotide array analysis.

In one embodiment, said determination of said methylation state in step ii) is or comprises the determination of a methylation score of said PITX2 gene or of one or several regulatory sequences thereof in said biological sample, and wherein prediction of said TNBC patient's response to said anthracycline-containing polychemotherapy is based on whether the methylation score in said biological sample exceeds a defined threshold methylation score, wherein said prediction is made in terms of one or several parameters selected from disease-free survival (DFS), metastasis-free survival (MFS) and overall survival (OS), and wherein said prediction is negative, if the methylation score of said sample is equal to or does not exceed said defined threshold methylation score, and wherein said prediction is positive, if said methylation score of said sample does exceed said defined threshold methylation score,

wherein said methylation score in said biological sample is determined according to the formula:

$$methylation score in biological sample = \frac{100}{1 + {2}^{( CT methylated - CT unmethylated )}}$$

wherein “CT methylated” is the cycle threshold value of methylated PITX2 and/or of methylated regulatory sequence(s) thereof,

and wherein “CT unmethylated” is the cycle threshold value of unmethylated PITX2 and/or of unmethylated regulatory sequence(s) thereof.

In one embodiment, the method comprises the steps:

i′) isolating genomic DNA from a biological sample taken from said patient and treating said genomic DNA, or a fragment thereof, with one or more reagents, in order to convert 5-position unmethylated cytosine bases to uracil or to another base that is detectably dissimilar to cytosine in terms of hybridization properties, thus producing treated genomic DNA; contacting said treated genomic DNA or said treated fragment thereof, with an amplification enzyme and at least two primers comprising, in each case, a contiguous sequence of at least 16 nucleotides in length that is complementary to, or hybridizes under stringent conditions to a sequence of the PITX2 gene and/or to one or several of its regulatory sequences thereof, and/or to complements thereof, wherein said at least two primers hybridize to said sequence(s) only if such sequence(s) has(have) been treated with said one or more reagents, such that the treated DNA or a fragment thereof is amplified to produce one or more amplificates, and wherein during amplification there are additionally at least two PITX2-specific probes present that distinguish between methylated and unmethylated PITX2 sequences, said at least two PITX2-specific probes producing two signals being indicative of methylated and unmethylated PITX2 sequences, respectively, said signals being proportional to the amount of methylated und unmethylated PITX2-sequences present in said biological sample, respectively,

ii′) determining, based on said signals of methylated und unmethylated PITX2-sequences a methylation score of said PITX2 gene in said biological sample,

wherein said methylation score in said biological sample is determined according to the formula

$$methylation score in biological sample = \frac{100}{1 + {2}^{( CT methylated - CT unmethylated )}}$$

wherein “CT methylated” is the cycle threshold value of methylated PITX2 and/or of methylated regulatory sequence(s) thereof,

and wherein “CT unmethylated” is the cycle threshold value of unmethylated PITX2 and/or of unmethyated regulatory sequence(s) thereof, and

iii′) making a prediction of said TNBC patient's response to anthracycline-containing polychemotherapy based on said determined methylation score in said biological sample, and wherein prediction of said TNBC patient's response to said anthracycline-containing polychemotherapy is based on whether the methylation score in said biological sample exceeds a defined threshold methylation score, wherein said prediction is made in terms of one or several parameters selected from disease-free survival (DFS), metastasis-free survival (MFS) and overall survival (OS), and wherein said prediction is negative, if the methylation score of said sample is equal to or does not exceed said defined threshold methylation score, and wherein said prediction is positive, if said methylation score of said sample is does exceed said defined threshold methylation score.

In one embodiment, the method further comprises

iv) determining a suitable treatment regimen for said patient, wherein such suitable treatment regimen for said patient is a treatment with polychemotherapy including one or several anthracyclines if said prediction of step iii) or iii') is positive, and said suitable treatment regimen is a therapy excluding anthracycline treatment, if said prediction is negative.

In one embodiment, said defined threshold methylation score is in the range of from 4-10, particularly in the range of from 5-8, more particularly 5-7, even more particularly 6-7, most particularly approximately 6.35+/−0.3.

In one embodiment, said biological sample is selected from the group consisting of breast cancer-derived tumor cell lines and breast cancer tissues, e.g. presurgical core biopsies, biopsies taken at time of primary surgery, circulating peripheral breast cancer tumor cells, tumor-afflicted lymph nodes, metastases, in particular in the frozen state or fixed with any fixative and then embedded in paraffin, bodily fluids such as nipple aspirate, blood, serum, plasma, urine or any other bodily fluid, and combinations thereof.

In one embodiment, the PITX2 gene has a sequence represented by SEQ ID NO:1 or SEQ ID NO:2.

In one embodiment, said sequence which said at least two primers are complementary to or hybridize thereto has a sequence represented by SEQ ID NO:3 and/or 4, and/or wherein said sequence of the PITX2 gene before treatment with said one or more reagent(s) has a sequence represented by SEQ ID NO:5, and/or wherein said at least two primers have sequences represented by SEQ ID NO: 6 and 7; and/or wherein said at least two PITX2-specific probes have sequences represented by SEQ ID NO:8 and SEQ ID NO:9.

In one embodiment, said at least two PITX2-specific probes are Taqman hydrolysis probes that distinguish between methylated and unmethylated PITX2 sequences, wherein a first Taqman hydrolysis probe is specific for methylated PITX2 sequence(s) and produces a first signal upon amplification of methylated PITX2 sequence(s), and wherein a second Taqman hydrolysis probe is specific for unmethylated PITX2 sequence(s) and produces a second signal different from said first signal, upon amplification of unmethylated PITX2 sequence(s), said first and said second signal preferably being fluorescence signals.

The objects of the present invention are also solved by the use of a PITX2 gene, a PITX2 regulatory sequence, or a stretch of at least 16 contiguous nucleotides of the foregoing sequences, or of a complementary sequence thereto or of a sequence that hybridizes under stringent conditions to any of the foregoing sequences, in a method for predicting a triple-negative breast cancer (TNBC) patient's response to anthracycline-containing adjuvant polychemotherapy according to the present invention.

In one embodiment, said PITX2 gene, said PITX2 regulatory sequence, said stretch of at least 16 contiguous nucleotides of the foregoing sequences, said complementary sequence thereto, or said sequence that hybridizes under stringent conditions to any of the foregoing sequences, is selected from any of the sequences represented by SEQ ID NO:1-9.

The present inventors have surprisingly found that, as opposed to non-triple-negative breast cancer (non-TNBC), a low DNA methylation state in the PITX2 gene, and thus a hypomethylation thereof, means that such a patient is likely to be non-responsive to anthracycline treatment. A TNBC patient thus diagnosed with a hypomethylation of the PITX2 gene is therefore likely not to respond to an anythracycline treatment and should therefore not be treated with anthracyclines, as this will have little or no positive effect on the patient's recovery. For such a patient, the treating physician will therefore choose another suitable treatment which does not involve the use of anthracyclines.

The term “methylation state”, as used herein, is meant to refer to the degree of methylation present in a nucleic acid of interest. This may be expressed in absolute or relative terms, i. e. as a percentage or other numerical value or by comparison to another tissue and may be described as “hypermethylated”, “hypomethylated” or as “having significantly similar or identical methylation status”. A “methylation score” as used therein, is a continuous variable and is typically expressed as “percent methylation ratio” (PMR). The “methylation score” is typically calculated according to the formula 100/(1+2^{(CTmethylated-CTunmethylated)})wherein “CT methylated” is the cycle threshold value of methylated sequence of interest, and wherein “CT unmethylated” is the cycle threshold value of the unmethylated sequence of interest. Cycle threshold values are a standard entity in quantitative PCT and refer to the number of cycles required for the signal to exceed background level. The term is a standard term and is known to a person skilled in the art. Cycle threshold values are inversely proportional to the amount of target nucleic acid in the sample, i. e. the lower the CT value is, the greater the amount of target nucleic acid in the sample. A methylation score can be calculated with or without normalization against one or several housekeeping genes. If such normalization is involved, this is also sometimes referred to as “double delta CT analysis”. In the experimental section that is described hereafter, cycle threshold values are determined automatically by the software of the quantitative PCR cycler. More specifically, in the experiments performed by the present inventors,

Cycle threshold values (CT) are determined automatically for the markers PITX2 methylated (FAM-Channel) and PITX2 unmethylated (VIC/HEX-Channel) separately by the qPCR cycler software by the course of the fluorescent signal readout during the PCR cycle program and including adaptive baseline correction and amplification-based threshold value (in the area of 5-60% total fluorescence level, averaged over technical replicates). Data transformation of CT values in a methylation score or, synonymously herewith, percent methylation ratio (PMR) values is facilitated by a modified 2exp^{ΔCT} method for a duplex probe system with internal calibration and standardization.

The term “threshold” or “defined threshold methylation score”, as used herein, is meant to refer to a specifically defined value or a specific (narrow) range of DNA methylation scores, which separates those TNBC patients likely to benefit from an anthracycline treatment from those TNBC patients which are not likely to benefit from such anthracycline treatment. If the methylation score of the PITX2 gene in a biological sample is equal or smaller than the defined threshold methylation score, then the patient will likely not benefit from an anthracycline treatment. According to one embodiment of the invention, such a defined threshold methylation score is in the range of from 4-10, preferably from 5-8, more preferably from 5-7, even more preferably from 6-7, most preferably around 6.35, e.g. 6.35±0.3.

The term “PITX2 gene” refers to the paired-like homeodomain transcription factor 2. An example embodiment of such PITX2 gene has the sequence represented by SEQ ID NO: 1 and can be found e. g. at Worldwide Website: ncbi.nlm.nih.gov/nuccore/161086966 or as entry Worldwide Website: entry:ncbi.nlm.nih.gov/nucleotide?cmd=Retrieve&dopt=GenBank&list uids=13183092 GenBank: AF238048.1 (SEQ ID NO:2).

### A Preferred PITX2 Sequence According to one Embodiment of the Invention is

Entrez gene ID 5308; Amplicon length 144; Reference sequence (Ref Seq) ID NT_016354.18; Detected CpG in Ref Seq 3 CpG in 36106573-36106600 which is shown below as SEQ ID NO:5 and which specifies the location of 3 CpG dinucleotides detected with Taqman hydrolysis probes, e.g. SEQ ID NO: 8 or 9.

### Exemplary Sequences in Accordance with the Present Invention

Bisulfite-converted PITX 2 sequence with a fully methylated status (SEQ ID NO:3) to which the Taqman probe for methylated sequences will anneal (e.g. SEQ ID NO:8):

gtaggggagggaagtagatgttagcgggtcgaagagtcgggagtcggag
tcgggagagcgaaaggagaggggatttggcggggtatttaggagttaat
cgaggagtaggagtacggatttttattgtggaaaggaggattagaa

Bisulfite-converted PITX2 sequence with an unmethylated status (SEQ ID NO:4) to which the Taqman probe for unmethylated sequences will bind (SEQ ID NO:9):

gtaggggagggaagtagatgttagtgggttgaagagttgggagttggag
ttgggagagtgaaaggagaggggatttggtggggtatttaggagttaat
tgaggagtaggagtatggatttttattgtggaaaggaggattagaa

Genomic (non-bisulfite-converted PITX2 sequence (SEQ ID NO: 5):

gcaggggagggaagcagatgccagcgggccgaagagtcgggagccggag
ccgggagagcgaaaggagaggggacctggcggggcacttaggagccaac
cgaggagcaggagcacggactcccactgtggaaaggaggaccagaa

### Exemplary Primer and Probe Sequences in Accordance with the Present Invention

PITX2-R
(SEQ ID NO: 6)
ttctaatcctcctttccacaataa
PITX2-F
(SEQ ID NO: 7)
gtaggggagggaagtagatgtt
PITX2-Pm
(SEQ ID NO: 8)
agtcggagtcgggagagcga
PITX2-Pu
(SEQ ID NO: 9)
agttggagttgggagagtgaaaggaga

F: forward primer

R: reverse primer

Pm: methylated probe (Taqman Hdyrolysis-probe with FAM)

Pu: unmethylated probe (Taqman Hdyrolysis-probe with VIC or HEX)

The term “anthracyclines”, as used herein, is meant to refer to a group of drugs obtained from Streptomyces bacteria, in particular Streptomyces peucitius. Examples of anthracyclines are daunorubicin, doxorubicin, epirubicin, idarubicin, valrubicin and mitoxantrone.

The term “PITX2 gene”, as used herein, is meant to refer to both introns and exons of the PITX2 gene as well as to regulatory sequences of PITX2. For example, such term also includes a promoter of the PITX2 gene. The term “PITX2 sequence”, as used herein, is meant to refer to any such sequence of PITX2. It may refer to partial sequences of the

PITX2 gene as short as 16 contiguous nucleotides or more. A probe or primer or sequence that is “PITX2-specific” is meant to refer to any primer or probe that specifically binds to, is complementary to or hybridizes to a PITX2-gene.

The term “hybridizes to” is meant to refer to a scenario wherein a single-stranded nucleic acid sequence binds to or anneals to another single-stranded nucleic acid. In one embodiment, this is achieved under a variety of conditions, e. g. stringent conditions. A person skilled in the art is able to determine stringent conditions for nucleic acid hybridization. For Array experiments: “Stringent hybridization conditions,” as defined herein, involve hybridizing at 68° C. in 5× SSC/5× Denhardt's solution/1.0% SDS, and washing in 0.2x SSC/0.1% SDS at room temperature, or involve the art-recognized equivalent thereof (e.g., conditions in which a hybridization is carried out at 60° C. in 2.5× SSC buffer, followed by several washing steps at 37° C. in a low buffer concentration, and remains stable). Moderately stringent conditions, as defined herein, involve including washing in 3× SSC at 42° C., or the art-recognized equivalent thereof. The parameters of salt concentration and temperature can be varied to achieve the optimal level of identity between the probe and the target nucleic acid. Guidance regarding such conditions is available in the art, for example, by Sambrook et al., 1989, Molecular Cloning, A Laboratory Manual, Cold Spring Harbor Press, N.Y.; and Ausubel et al. (eds.), 1995, Current Protocols in Molecular Biology, (John Wiley & Sons, N.Y.) at Unit 2.10.

For Taqman qPCR systems the inventors would recommend hybridizing at 60° C. in a commercial quantitative PCR buffer (e.g. Quantitect Probe PCR buffer from the Company Qiagen) including reaction buffer, dNTPs, Magnesium and TAQ Polymerase.

The term “triple-negative breast cancer (TNBC)”, as used herein, is meant to refer to a type of breast cancer which is characterized by a lack of estrogen receptor (ER) and progesterone receptor (PgR) expression and a low expression of human epidermal growth factor receptor 2 (HER2). (preferably in accordance with a Dako IHC score 0-2, without amplification of the HER2 gene assessed by fluorescence in situ hybridisation according to the Sankt Gallen guidelines for breast cancer.)

The term “predicting a patient's response to a treatment”, as used herein, is meant to refer to a prediction of the outcome of a particular treatment, if performed on a patient. Preferably, such a statement about the response or outcome of a particular treatment is made in terms of one or several parameters selected from disease-free survival (DFS), metastasis-free survival (MFS) and overall survival (OS).

The term “regulatory sequence of a gene”, as used herein, is meant to refer to a sequence which affects the expression of a gene. Such a regulatory sequence may be located within, proximal or distal to said gene. A regulatory sequence includes, but is not limited to constitutive promoters, tissue-specific promoters, developmental-specific promoters, inducible promoters and the like. Promoter-regulatory elements may also include enhancer sequence elements that control transcriptional or translational efficiency of a gene.

The term “methylation” refers to the presence or absence of 5-methyl-cytosine (5-mCyt)” at one or a plurality of CpG dinucleotides within a DNA sequence.

The term “methylation state”, as used herein, is meant to refer to the degree of methylation present in a nucleic acid of interest, e.g. the PITX2 gene. This may be expressed in absolute or relative terms, i.e. as a percentage or other numerical value or by comparison to another tissue and may therein be described as “hypermethylated, hypomethylated or as having significantly similar or identical methylation status”.

The methylation score is a continuous variable (and is herein also sometimes referred to as “Percent methylation ratio”), Methylation state discerns between a high methylated (hypermethylated) or low methylated (hypomethylated) state for the PITX2 gene according to a specific cut off value (e.g. 6.35 PMR).

The term “Taqman hydrolysis probe” as used herein, is meant to refer to an oligonucleotide probe which is sequence specific and which has a dye attached to one end of the probe and a quencher at the other end of the probe. Typically, the dye is a fluorescent dye, and the quencher is a fluorescence quencher. In the context of embodiments of the present invention, the oligonucleotide probe is PITX2-specific, in particular specific for methylated PITX2 sequences or for unmethylated PITX2 sequences.

In the Taqman probe, the quencher molecule quenches the fluorescence emitted by the fluorophor via fluorescence resonance energy transfer, for as long as the fluorophor and the quencher are in proximity to each other. During amplification, the amplification enzyme, typically a Taq polymerase, degrades the oligonucleotide probe due to the exonuclease activity of the polymerase, and a degradation of the probe releases the dye, typically the fluorophor, from the probe, as a result of which the quenching no longer occurs. The signal detected is thus directed proportional to the number of amplificates which, in turn, is directly proportional to the number of copies originally present in the biological sample.

Typical examples of fluorophor that are used in Taqman hydrolysis probes are fluorescein derivatives (FAM), VIC dye, HEX dye, i. e. 5′-hexachloro-fluorescein).

The term “adjuvant” chemotherapy, in the context of breast cancer, is meant to refer to a chemotherapy that is performed after surgery or any other form of primary therapy. The term “polychemotherapy” as used herein, is meant to refer to a chemotherapy by several agents. These agents may be administered sequentially, concomitantly, in one or several doses.

### EXAMPLES

### Example 1

### Patient Collective

The inclusion criteria for the current retrospective study were patients with histologically confirmed invasive triple-negative breast cancer, with no signs of distant metastasis at the time of diagnosis, availability of sediment pellets after protein extraction from tumour tissues (as a source for DNA; for methodology see Ref 31), availability of follow-up data and appropriate written informed patient consent. Sediment pellets of 95 patients meeting these predefined criteria were obtained from the Tumour Bank and Mamma CA Database of the Klinikum rechts der Isar, stored at the Department of Obstetrics and Gynecology of the Technical University of Munich. All 95 patients were treated at the Klinikum rechts der Isar, Munich, Germany, between 1991 and 2006. Study approval was obtained from the Ethics Committee of the Medical Faculty of the Technical University of Munich, Germany. Median age of the patients at time of diagnosis was 59 months (range: 27-96 months) and the median follow-up time 79 months (range 8-216 months).

The inventors aimed for a minimum follow-up period of 60 months for those patients who did not suffer from any events. For five patients this was not achieved since they did not want to participate in any further follow-up examinations. All 95 patients underwent surgical treatment (65 patients: breast conserving therapy, 30 patients: mastectomy). 77 patients received radiotherapy. 23 patients did not receive any chemotherapy, 72 patients were treated with adjuvant chemotherapy. In the majority of cases (n=56) and anthracycline-containing chemotherapy regimen was applied (Table 1). Histomorphological and clinical characteristics are depicted in Table 2.

### Cell Lines and Fresh-Frozen Tissues

Two fresh-frozen breast cancer tissues obtained from the Tumour Bank of the Department of Obstetrics and Gynecology (Klinikum rechts der Isar, Munich, Germany). The breast cancer cell lines MCF-7 and MDA-MB-231 (CLS Cell Lines Service GmbH, Eppelheim, Germany) were employed for preliminary testing and quality assessment.

### DNA Extraction

Unless otherwise stated, all the reagents, protocol sheets and materials applied in this study were obtained from Qiagen (Hilden, Germany). Fresh-frozen breast cancer specimens were obtained from the Tumour Bank of the Department of Obstetrics and Gynecology (Klinikum rechts der Isar, Munich, Germany)and processed accordingly.[31] DNA extraction from sediment pellets was performed following the QiAamp DNA Mini and Blood Mini Handbook protocol. Lysis was performed manually, followed by semi-automated extraction with the QIAcube system. Approximately 30 mg of sediment pellet was used. The extracted DNA was stored at −20° C.

### DNA Concentration Determination and Adjustment

The Nano Drop 2000c spectrophotometer with appropriate software (Nanodrop/Thermo Fisher Scientific, Wilmington, USA) was used for the determination of the DNA concentration. For PITX2 methylation score measurement optimisation, For each sample, 310 ng of DNA was used in the subsequent bisulfite conversion step. For 13 samples, a DNA concentration step with the Speed Vac System Concentrator 5301 (Eppendorf, Hamburg, Germany) had to be performed.

### Bisulfite Conversion

Bisulfite conversion was performed according to the EpiTect Bisulfite Handbook, employing the ABI PCR Cycler (Applied Biosystems, Darmstadt, Germany) (Program details: 1^{st} 5 min at 99° C., 2^{nd} 25 min at 60° C., 3^{rd} 5 min at 99° C., 4^{th} 85 min at 60° C., 5^{th} 5 min at 99° C., 6^{th} 175 min at 60° C.). Clean-up of the bisulfite converted DNA was carried out in a semi-automated procedure according to the EpiTect Bisulfite Kit protocol sheet

### Quantitative Real Time PCR

PITX2 primers and probes for the methylated and unmethylated DNA status in a duplex system, provided by Qiagen were used (Table 4). qPCR was carried out according to the provider protocol (EpiTect MethyLight Assay Hs_PITX2) using an ABI 7000 Taqman System (Applied Biosystems, Darmstadt, Germany) (Run details: 1^{st} 15 min at 95° C.; 2^{nd} 48 cycles comprising of 15 sec at 95° C. and 1 min at 60° C.) and the Quantitect 2× QPCR Mastermix (Qiagen, Hilden, Germany) in a final volume of 20 pl. Each specimen was measured in triplicates. A minimum of two runs per specimen was performed. On each plate, a positive control comprising of fully methylated bisulfite-converted human control DNA, a negative control (RNAse-free water) and 7.75 ng of bisulfite-converted MCF-7 DNA were included.

Statistics and Quality Assessment Reporting of this study was carried out observing the REMARK criteria.[22] For calculation of PITX2 DNA methylation scores, a modified 2exp-ΔΔCT-method was used as described earlier (Ref 11). CT mean values of triplicates were calculated for the methylated and unmethylated state, which were used for calculation of the final methylation scores. For quality reasons, results with both CT values (methylated and unmethylated) greater than 38 cycles were excluded, in this case the measurements had to be repeated. Mean values, standard deviations and coefficients of variation of the different qPCR runs were calculated.

In case the coefficient of variation exceeded 0.3, a third qPCR run was carried out and the results calculated accordingly. Primary endpoints were defined as overall survival (OS), metastasis-free survival (MFS; time to detection of distant metastasis) and disease-free survival (DFS; period after primary disease elimination in which no disease was detected). The date of primary surgery was considered as the follow-up index date.

In order to discriminate low- and high-risk patients with regards to DFS, MFS and OS, optimised cut-off values were calculated with the “maximum-selected log-rank statistic” using the maxstat.test function as implemented from the program library “maxstat” of the program “R” (R Development Core Team 2012).[14, 35] Kaplan-Meier analyses were carried out to estimate and display empirical survivor functions for OS, MFS and DFS. The Logrank test was used for calculating the respective p-values. Cox regression models were used for univariate estimation of hazard ratios (HRs) with regards to DFS, MFS and OS. Due to the limited event numbers, multivariable analysis was carried out in an exploratory way. Furthermore, covariates were added stepwise to the variable PITX2 methylation scores and the according hazard rations (HR) and 95% confidence intervals were depicted in Forest plot diagrams in order to test whether PITX2 methylation adds independent additional information to the other factors.

### Example 2

### Detailed Results of Preliminary Tests

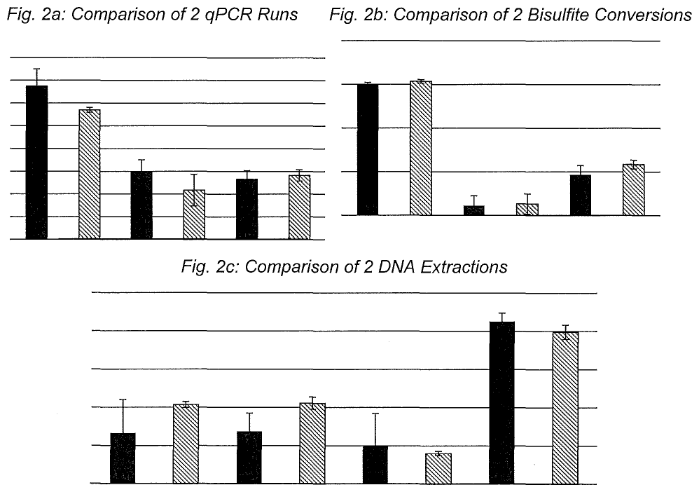

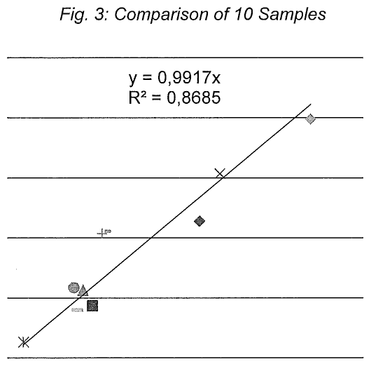

In order to evaluate the impact of the different processing steps (DNA extraction, bisulfite conversion and qPCR run), a preliminary test consisting of three different levels was accomplished (Table 5 and FIGS. 2 and 3). Ten samples of the triple-negative patient collective were chosen randomly. Whether diverse DNA methylation results derive from the same bisulfite converted DNA measured in two separate qPCR runs was examined on the first level. For three specimens, the substrates which were applied to the two distinct qPCR runs derived from the same DNA extraction and bisulfite conversion, leaving the qPCR run the only variable factor (FIG. 2a). For each sample, the coefficient of variation regarding the DNA methylation scores measured in two different qPCR runs was calculated. The mean value of these coefficients of variation was 0.09. On the second level, the impact of bisulfite conversion was examined using three other samples. This time, the substrate of one sample derived from the same DNA extraction but both bisulfite conversion and qPCR constituted differing factors which might possibly influence the score results (FIG. 2b). The mean value of the coefficients of variation of the measured methylation scores was determined 0.07. Finally, four different specimens underwent an experiment set-up in which DNA extraction, bisulfite conversion as well as qPCR runs were different for the two reagents measured per each sample. 0.14 was the calculated mean value of coefficients of variation (FIG. 2c). When plotting the results of the two different runs per each of the ten samples, a high correlation (R>0.93) between the two PITX2 DNA methylation scores was observed with a mean value of coefficient of variation of 0.10 (FIG. 3).

Score reproducibility was demonstrated through gradual and serial dilution series and subsequent qPCR runs using 7.75 ng of bisulfite-converted DNA extracted from the MCF-7 cell line and two fresh-frozen breast cancer tissues (Tissue X and Y; Table 6). By serial dilutions, the applied bisulfite converted DNA concentration was bisected with each dilution step (6 steps were carried out resulting in concentrations of 100/50/25/12.5/6.25/3.125/1.5625%), whereas through gradual dilution the original concentration was reduced by 20% with each of the 4 steps (leading to concentrations of 100/80/60/40/20%). For the MCF-7 cell line, one serial and two gradual dilutions were accomplished, whereas the fresh-frozen breast cancer tissues each underwent one gradual dilution. The mean value and coefficient of variation of the different dilution steps was calculated for each dilution series ranging between 0.00 and 0.06 (Table 6).

For quality assurance of the different qPCR runs, positive controls, negative controls and the MCF-7 cell line were included in each run, whereas another control cell line with median PITX2 DNA methylation levels, MDA-MB-231, was only included in 27.3% of all runs. For the PITX2 DNA methylation scores of the different runs and controls, the respective mean values and coefficients of variation were calculated (Table 7). Stable scores for the controls were obtained throughout the different qPCR runs.

### Example 3

### Preliminary Tests

Preliminary tests were carried out in order to assess the assay stability (FIG. 2-3 and Tables 5-7). Score reproducibility was demonstrated through gradual and serial dilution series and subsequent qPCR runs using 7.75 ng of bisulfite-converted DNA extracted from MCF-7 cells and two fresh-frozen breast cancer tissues (Tissue X and Y; Table 6). The calculated low coefficients of variation (highest 0.06; Table 6) indicate that even with low concentrations of bisulfite-converted DNA, PITX2 methylation scores can be determined reliably. For further quality assurance of the different qPCR runs, positive controls, negative controls and the MCF-7 cell line were included in each run. The according low coefficients of variation (highest 0.08; Table 7) indicate that stable scores could be obtained throughout the different qPCR runs. Comparison of methylation scores of ten randomly chosen triple-negative samples, which were processed in different turns (Table 5 and FIG. 2/3), revealed a low median coefficient of variation of 0.10 and high correlation (R>0.93; FIG. 3).

PITX2 DNA Methylation Scores, Clinical Outcome and Subgroup Definition Valid PITX2 DNA methylation scores were obtained for all patients. The median methylation score was 10.05. Approximately 75.3% of the patients survived 5 years; the estimated 5-year DFS probability was 74.6% and the estimated 5-year MFS probability 80.3%. The majority of events occurred within 5 years (92.0% of disease recurrences, 90.0% of metastases and 92.0% of deaths). To analyse whether PITX2 methylation might constitute a prognostic or predictive marker in TNBC, subgroup analyses were performed. According to the adjuvant treatment, three different subgroups were defined: no-chemotherapy subgroup (NCS), non-anthracycline-based chemotherapy subgroup (NABCS) and anthracycline-based chemotherapy subgroup (ABCS). For detailed clinical and histomorphological characteristics see Table 2.

### Cut-Off Definition and Application

Due to the fact that an application of the PITX2 DNA methylation cut-off value defined by Harbeck et al of 22.9 [11] did not lead to any significant risk group separation, the ‘maxstat.test’ R-function was used in order to search for optimised cut-off values. Using this conservative test, which already accounts for the issue of multiple testing, no statistical significant cut-off value was found when analysing the whole TNBC collective (DFS: cut-off 6.35, p=0.529; MFS cut-off 6.35, p>0.99; OS cut-off 7.34, p=0.611). As therapy based subgroup analyses were of special interest to the inventors, the search for optimised cut-off values was also performed in the ABCS, NABCS and NCS. In the anthracycline-based chemotherapy subgroup (ABCS) a cut-off value of 6.35 was calculated for the three primary endpoints. Only for DFS the risk group separation within the anthracycline-based chemotherapy subgroup was found to be statistically significant (DFS p=0.015; OS p=0.091; MFS p=0.275). Neither for the NABCS nor the NCS, statistical significant cut-off values could be defined.

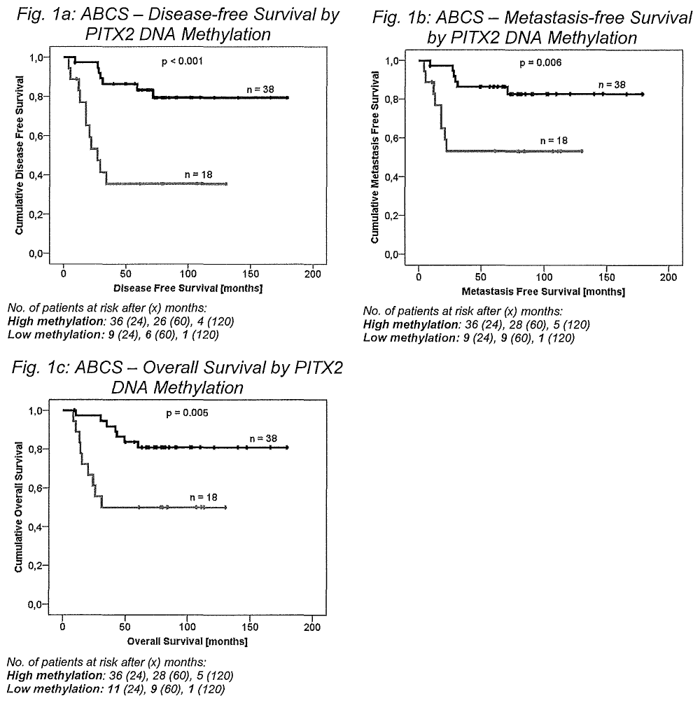

In the ABCS, Kaplan Meier analyses were carried out using the defined cut-off value of 6.35 for the endpoints DFS, MFS and OS in order to estimate and display empirical survival tendencies. Low methylation was associated with statistically significant worse prognosis regarding DFS (p<0.001, 5-year DFS 35.6% vs. 83.5%, FIG. 1a), MFS (p =0.006, 5-year DFS 53.3% vs 86.5%, FIG. 1b) and OS (p=0.005, 5-year DFS 50.0% vs 80.9%, FIG. 1c).

### ABCS—Uni- and Multivariable Cox Regression Analysis

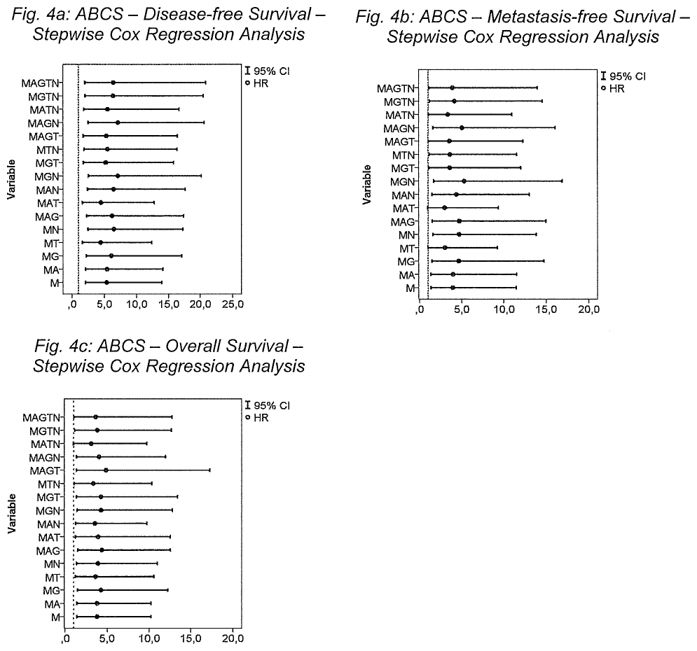

In order to evaluate the impact of PITX2 DNA methylation scores on clinical outcome in the ABCS, uni- and multivariable Cox regression analyses were carried out for the different endpoints. Due to the limited events numbers, multivariable analysis was only carried out with exploratory intentions. Therefore, stepwise inclusion of the established clinical/histomorphological factors age, tumor grading (G3 vs. G^{1/2}), tumour size (>2 vs.≤2cm) and nodal status (positive vs. negative) as covariates was necessary when testing whether PITX2 DNA methylation (≤6.35 vs.>6.35) constitutes a stable and independent variable. The calculated hazard ratios of PITX2 DNA methylation obtained from stepwise addition of covariates for the three endpoints were stable and with regards to DFS each of them was statistically significant (FIG. 4a). However, two 95% confidence intervals exceeded the value of 1.0 when analysing MFS and one when analysing OS, leading to statistical insignificance (FIGS. 4b and 4c). Both in univariate (DFS: HR=5.36, 95% CI 2.06-13.95; MFS: HR=3.95, 95% CI 1.36-11.46; OS: HR=3.78, 95% CI 1.40-10.20) and multivariable (DFS: HR=6.40, 95% CI 1.96-20.88; MFS: HR=3.89, 95% CI 1.09-13.95; OS: HR=3.62, 95% CI 1.03-12.72) Cox regression, PITX2 DNA methylation was found to contribute significant additional information with regards to the three endpoints (Table 3). Out of the established factors, only age contributed significant information in univariate analysis of OS (HR=1.05, 95% CI 1.01-1.10).

### Preliminary Tests and Quality Assessment

To the inventors' knowledge, the current study is the first to analyse PITX2 DNA methylation of TNBC DNA obtained from sediment pellets. Other research groups who examined this marker in non-TNBC used e.g. FFPE tissue sections or fresh-frozen tumour specimens as primary material.[11, 12, 21, 25] On this basis, preliminary tests had to be carried out in order to prove applicability of sediment pellets for measuring DNA methylation markers and to show that the achieved PITX2 scores are reliable and reproducible. High standards of quality criteria were applied. In accordance with the criteria used in the multicenter study of

Harbeck et al.[11], measurements with both methylated and unmethylated CT values greater than 38 were excluded. Additionally, the quality criteria was extended since for each sample the coefficient of variation of two different qPCR results was calculated and in case of it exceeding 0.3 another qPCR run was carried out. The impact of sample processing techniques on obtained PITX2 DNA methylation scores was examined (Example 2 above).

Low mean values of coefficient of variation (0.09, 0.07 and 0.14 respectively) and a high correlation between PITX2 DNA methylation scores using material from the same samples processed in different turns were revealed (r>0.93). This indicated that the influence of processing techniques on PITX2 DNA methylation scores can be neglected. Stable measurements gained from dilution series showed that even when applying small amounts of bisulfite converted DNA, robust results can be achieved with the used PITX2 primer and probe system. These results indicate a reliable workflow and provide evidence for the use of PITX2 DNA methylation score obtained from sediment pellets as a robust marker.

### Clinical Outcome, Clinical Data and PITX2 DNA Methylation Score

The estimated 5-year OS rate (75.3%) was similar to the 5-year OS rate of the triple-negative cohort analysed by Bauer et al (77%).[5] In concordance with the reported bad prognosis [23, 34, 36] of triple-negative diseases, the relatively low 5-year MFS (80.3%) and DFS rates (74.6%) in the current collective were not surprising. The tendency of TNBC towards early onset[1], higher grade[2] and larger size[7, 18, 37] was obvious in the current collective as the median age at time of diagnosis was 59, 82.1% of examined tumours were of grade 3 and 65.3% were found to be larger than 2.0 cm in diameter. With 45.2% of patients being nodal positive, lymph node involvement was relatively common.

The median PITX2 methylation score of the analysed TNBC was 10.05, considerably lower than in non-TNBC.[11, 21, 25]. In different types of tumours, PITX2 expression levels seem to have different consequences and aberrant methylation levels were found in various malignant cancers. Although no study revealed a direct association between PITX2 hypomethylation and tumourigenesis, PITX2 over-expression was found in several cancer types such as non-functional pituitary adenomas[24] and ovarian cancer.[8] On the other hand, PITX2 downregulation and hypermethylation was present in e.g. colorectal[13] and prostate tumours.[3] These results indicate that PITX2 might possess a dose-dependent oncogenic potential in different tissues and that one of the factors disturbing its physiological balance might be epigenetic deregulation via DNA methylation.

### PITX2 DNA Methylation as Predictive Marker in TNBC

To the inventors' knowledge, this is the first study examining whether PITX2 DNA methylation can serve as a predictive marker in triple-negative breast cancer. As already mentioned, several previous studies examined PITX2 DNA methylation in non-TNBC and found that high DNA methylation scores were associated with poor clinical outcome (Table 8). In a study by Hartmann et al on estrogen receptor-positive, nodal-positive breast cancer patients who had received an adjuvant anthracycline-based chemotherapy, high PITX2 DNA methylation was associated with poor DFS, MFS and OS.[12] Contrary to its role in non-TNBC, low PITX2 methylation seems to be a statistically independent marker, predicting response to anthracyclines in a TNBC collective whereas no association between PITX2 DNA methylation and clinical outcome in the NABCS and NCS was found.

In the ABCS, low PITX2 DNA methylation was associated with poor DFS, MFS and OS. Uni- and multivariable Cox analysis as well as stepwise inclusion of covariates suggested PITX2 DNA methylation as a statistically independent and stable factor. Advanced age was found to be the only established prognostic factor which was marginally associated with poor OS in univariate Cox analysis.

The current results, which indicate a reverse relationship between PITX2 DNA methylation and outcome in anthracycline-receiving TNBC compared to non-TNBC, are surprising.

However, they lend support to PITX2 DNA methylation being a highly valuable marker as it can influence therapeutic decisions in triple-negative breast cancer. In TNBC, PITX2 DNA methylation is useful in order to identify patients who derive sufficient treatment through anthracyclines. Furthermore, the resistant subgroup with low methylation scores might need more intensified therapy or simply different treatment (e.g. PARP inhibitors, platinum-based chemotherapies or CMF) and thus should be spared the negative side-effects of anthracyclines.

TABLE 1
Adjuvant Chemotherapy Regimens

| | No. of Patients | |
|---|---|---|
| Regimen | (N = 72) | % |
| CMF | 16 | 22.2 |
| FEC | 21 | 29.2 |
| EC | 12 | 16.7 |
| EC + CMF | 3 | 4.2 |
| Anthracycline-plusTaxane- | 9 | 12.5 |
| Containing Polychemotherapy | | |
| Other Anthracycline-cContaining | 11 | 15.3 |
| Polychemotherapy | | |

TABLE 2
Patients’ Clinical and Histomorphological Characteristics

| | TNBC | | ABCS | | NABCS | | NCS | |
|---|---|---|---|---|---|---|---|---|
| | No. of | | No. of | | No. of | | No. of | |
| | Patients | | Patients | | Patients | | Patients | |
| Characteristic | (N = 95) | % | (N = 56) | % | (N = 16) | % | (N = 23) | % |
| Tumour Grade | | | | | | | | |
| 1 | 6 | 6.3 | — | — | — | — | 6 | 26.1 |
| 2 | 9 | 9.5 | 4 | 7.1 | 2 | 12.5 | 3 | 13.0 |
| 3 | 78 | 82.1 | 50 | 89.3 | 14 | 87.5 | 14 | 60.9 |
| No Data | 2 | 2.1 | 2 | 3.6 | — | — | — | — |
| Nodal Status | | | | | | | | |
| pN0 | 50 | 52.6 | 27 | 48.2 | 9 | 56.3 | 14 | 60.9 |
| pN1 | 33 | 34.7 | 20 | 35.7 | 7 | 43.8 | 6 | 26.1 |
| pN2 | 6 | 6.3 | 6 | 10.7 | — | — | — | — |
| pN3 | 4 | 4.2 | 2 | 3.6 | — | — | 2 | 8.7 |
| No Data | 2 | 2.1 | 1 | 1.8 | — | — | 1 | 4.3 |
| Tumour Size (cm) | | | | | | | | |
| ≤2 | 29 | 30.5 | 18 | 32.1 | 6 | 37.5 | 5 | 21.7 |
| >2 | 62 | 65.3 | 36 | 64.3 | 9 | 56.3 | 17 | 73.9 |
| No Data | 4 | 4.2 | 2 | 3.6 | 1 | 6.3 | 1 | 4.3 |
| Age at Time of | | | | | | | | |
| Diagnosis, Years | | | | | | | | |
| <50 | 67 | 70.5 | 20 | 35.7 | 4 | 25.0 | 4 | 17.4 |
| ≥50 | 28 | 29.5 | 36 | 64.3 | 12 | 75.0 | 19 | 82.6 |
| Histological Subtype | | | | | | | | |
| Invasive Ductal | 66 | 69.5 | 42 | 75.0 | 13 | 81.3 | 11 | 47.8 |
| Medullary | 9 | 9.5 | 7 | 12.5 | 1 | 6.3 | 1 | 4.3 |
| Lobular | 4 | 4.2 | 1 | 1.8 | 1 | 6.3 | 2 | 8.7 |
| Tubular | 2 | 2.1 | — | — | — | — | 2 | 8.7 |
| Others | 13 | 13.7 | 6 | 10.7 | 1 | 6.3 | 6 | 26.1 |
| No Data | 1 | 1.1 | — | — | — | — | 1 | 4.3 |
| Disease Recurrence | | | | | | | | |
| Yes | 25 | 26.3 | 18 | 32.1 | 1 | 6.3 | 6 | 26.1 |
| Median Time to | 27 | Months | 24.5 | Months | 51 | Months | 31.5 | Months |
| Disease Recurrence | (Range 4-101) | | (Range 4-72) | | | | (Range 9-101) | |
| No | 70 | 73.7 | 38 | 67.9 | 15 | 93.8 | 17 | 73.9 |
| Deceased | | | | | | | | |
| Yes | 25 | 26.3 | 16 | 28.6 | 1 | 6.3 | 8 | 34.8 |
| Median Time to Death | 31 | Months | 25 | Months | 55 | Months | 43.5 | Months |
| | (Range 8-70) | | (Range 8-60) | | | | (Range 8-70) | |
| No | 70 | 73.7 | 40 | 71.4 | 15 | 93.8 | 15 | 65.2 |
| Metastasised | | | | | | | | |
| Yes | 20 | 21.1 | 14 | 25.0 | 1 | 6.3 | 5 | 21.7 |
| Median Time to | 25.5 | Months | 19.5 | Months | 51 | Months | 39 | Months |
| Metastasis | (Range 4-101) | | (Range 4-71) | | | | (Range 24-101) | |
| No | 75 | 78.9 | 42 | 75.0 | 15 | 93.8 | 18 | 78.3 |

TABLE 3
ABCS Uni- and Multivariable Cox Regression Analyses

| | | | | |
|---|---|---|---|---|
| Variable | Hazard ratio | 95% CI | P | N |

DFS - Univariate Analysis

| | | | | |
|---|---|---|---|---|
| Age | 1.02 | 0.98-1.06 | 0.415 | 56 |
| Tumour Grading | 1.30 | 0.17-9.83 | 0.798 | 54 |
| Tumour Size | 2.75 | 0.80-9.52 | 0.109 | 54 |
| Nodal Status | 1.55 | 0.60-4.00 | 0.365 | 55 |
| PITX2 DNA | 5.36 | 2.06-13.95 | 0.001 | 56 |
| Methylation | | | | |

DFS - Multivariable Analysis

| | | | | |
|---|---|---|---|---|
| Age | 1.00 | 0.96-1.05 | 0.981 | 51 |
| Tumour Grading | 0.86 | 0.10-7.78 | 0.893 | 51 |
| Tumour Size | 1.13 | 0.26-5.01 | 0.871 | 51 |
| Nodal Status | 2.04 | 0.61-6.84 | 0.249 | 51 |
| PITX2 DNA | 6.40 | 1.96-20.88 | 0.002 | 51 |
| Methylation | | | | |

MFS - Univariate Analysis

| | | | | |
|---|---|---|---|---|
| Age | 1.03 | 0.98-1.08 | 0.209 | 56 |
| Tumour Grading | 0.94 | 0.12-7.2 | 0.952 | 54 |
| Tumour Size | 3.25 | 0.73-14.53 | 0.123 | 54 |
| Nodal status | 1.78 | 0.60-5.31 | 0.301 | 55 |
| PITX2 DNA | 3.95 | 1.36-11.46 | 0.011 | 56 |
| Methylation | | | | |

MFS - Multivariable Analysis

| | | | | |
|---|---|---|---|---|
| Age | 1.02 | 0.96-1.07 | 0.546 | 51 |
| Tumour Grading | 0.52 | 0.05-5.15 | 0.577 | 51 |
| Tumour Size | 1.78 | 0.29-10.88 | 0.531 | 51 |
| Nodal Status | 1.61 | 0.44-5.88 | 0.471 | 51 |
| PITX2 DNA | 3.89 | 1.09-13.95 | 0.037 | 51 |
| Methylation | | | | |

OS - Univariate Analysis

| | | | | |
|---|---|---|---|---|
| Age | 1.05 | 1.01-1.10 | 0.032 | 56 |
| Tumour Grading | 1.09 | 0.14-8.30 | 0.933 | 54 |
| Tumour Size | 1.62 | 0.52-5.02 | 0.404 | 54 |
| Nodal Status | 1.46 | 0.52-4.11 | 0.471 | 55 |
| PITX2 DNA | 3.78 | 1.40-10.20 | 0.009 | 56 |
| Methylation | | | | |

OS - Multivariable Analysis

| | | | | |
|---|---|---|---|---|
| Age | 1.05 | 0.99-1.11 | 0.092 | 56 |
| Tumour Grading | 0.54 | 0.06-5.23 | 0.594 | 56 |
| Tumour Size | 1.12 | 0.23-5.58 | 0.886 | 56 |
| Nodal Status | 0.99 | 0.30-3.21 | 0.982 | 56 |
| PITX2 DNA | 3.62 | 1.03-12.72 | 0.045 | 56 |
| Methylation | | | | |

TABLE 4
Specifications Regarding PITX2 Probe and Primer System
(According to Provider)

| | |
|---|---|
| Entrez gene ID | 5308 |
| Amplicon length | 144 |
| Reference sequence (Ref Seq) ID | NT_016354.18 |
| Detected CpG in Ref Seq | 3 CpG in 36106573-36106600 |

TABLE 5
Layout Plan of Preliminary Test data for qPCR robustness assessment

| | | No. of | DNA | Bisulfite | qPCR |
|---|---|---|---|---|---|
| Level | Comparison of | samples | extraction | conversion | run |
| 1 | 2 Different qPCR Runs | 3 | Same | Same | Different |
| 2 | 2 Different Bisulfite | 3 | Same | Different | Different |
| | Conversions | | | | |
| 3 | 2 Different DNA | 4 | Different | Different | Different |
| | Extractions | | | | |

TABLE 6
Dilution Series of MCF-7, Tissue X and Tissue Y and According Mean
Values & Coefficients of Variation

| | | No. of | | |
|---|---|---|---|---|
| | | Replicates | | |
| Dilution | Type of | per Dilution | Mean | Coefficient |
| Series of | Dilution | Step | value | of Variation |
| MCF-7 | Serial | 3 | 96.9 | 0.00 |
| MCF-7 | Gradual | 3 | 92.9 | 0.01 |
| MCF-7 | Gradual | 3 | 95.7 | 0.00 |
| Tissue X | Gradual | 3 | 66.9 | 0.04 |
| Tissue Y | Gradual | 3 | 44.6 | 0.06 |

TABLE 7
Positive Control, Negative Control, MCF-7 and MDA-MB-231 and
According Mean Values & Coefficients of Variation

| | | | | Coefficient |
|---|---|---|---|---|
| | No. of Replicates | No. of | Mean | of |
| Control | per qPCR Run | Experiments | Value | Variation |
| Positive Control | 3 | 22 | 92.3 | 0.01 |
| Negative Control | 2 | 22 | — | — |
| MCF-7 | 3 | 22 | 94.7 | 0.01 |
| MDA-MB-231 | 3 | 6 | 70.4 | 0.08 |

TABLE 8
Association Between PITX2 Methylation and Outcome in Non-TNBC

| | | High PITX2 |
|---|---|---|
| | | Methylation |
| Research Group | Patient Collective | Associated with |
| Maier et al [21] | HR+, N−, Adjuvant | Poor MFS |
| | Tamoxifen Monotherapy | |
| Nimmrich et al [25] | HR+, N−, Untreated | Poor MFS and OS |
| Harbeck et al [11] | ER and/or PR+, N−, | Poor MFS |
| | Adjuvant Tamoxifen | |
| | Monotherapy | |
| Hartmann et al [12] | ER+, N+, Adjuvant | Poor MFS, DFS, |
| | Anthracycline-based | OS |
| | Polychemotherapy | |

### REFERENCES

1. Krebs in Deutschland 2007/2008. 8. Ausgabe. Robert Koch-Institut (Hrsg) and die Gesellschaft der epidemiologischen Krebsregister in Deutschland e.V. (Hrsg). Berlin, 2012.

2. Albergaria A, Ricardo S, Milanezi F, Carneiro V, Amendoeira I, Vieira D, Cameselle-Teijeiro J, Schmitt F (2011) Nottingham Prognostic Index in triple-negative breast cancer: a reliable prognostic tool? BMC cancer 11:299. doi: 10.1186/1471-2407-11-299.

3. Banez L L, Sun L, van Leenders G J, Wheeler T M, Bangma C H, Freedland S J, Ittmann M M, Lark A L, Madden J F, Hartman A, Weiss G, Castanos-Velez E (2010) Multicenter clinical validation of PITX2 methylation as a prostate specific antigen recurrence predictor in patients with post-radical prostatectomy prostate cancer. The Journal of urology 184: 149-156. doi: 10.1016/j.juro.2010.03.012.

4. Basu M, Roy S S (2013) Wnt/beta-catenin pathway is regulated by PITX2 homeodomain protein and thus contributes to the proliferation of human ovarian adenocarcinoma cell, SKOV-3. The Journal of biological chemistry 288: 4355-4367. doi: 10.1074/jbc.M112.409102.

5. Bauer K R, Brown M, Cress R D, Parise C A, Caggiano V (2007) Descriptive analysis of estrogen receptor (ER)-negative, progesterone receptor (PR)-negative, and HER2-negative invasive breast cancer, the so-called triple-negative phenotype: a population-based study from the California cancer Registry. Cancer 109: 1721-1728. doi: 10.1002/cncr.22618.

6. Dent R, Trudeau M, Pritchard KI, Hanna W M, Kahn H K, Sawka C A, Lickley L A, Rawlinson E, Sun P, Narod S A (2007) Triple-negative breast cancer: clinical features and patterns of recurrence. Clin Cancer Res 13: 4429-4434. doi: 10.1158/1078-0432.CCR-06-3045.

7. Elias A D (2010) Triple-negative breast cancer: a short review. American journal of clinical oncology 33: 637-645. doi: 10.1097/COC.0b013e3181b8afcf. 8. Fung F K, Chan D W, Liu V W, Leung T H, Cheung A N, Ngan H Y (2012) Increased expression of PITX2 transcription factor contributes to ovarian cancer progression. PloS one 7: e37076. doi: 10.1371/journal.pone.0037076.

9. Geyer F C, Lacroix-Triki M, Savage K, Arnedos M, Lambros M B, MacKay A, Natrajan R, Reis-Filho J S (2011) beta-Catenin pathway activation in breast cancer is associated with triple-negative phenotype but not with CTNNB1 mutation. Modern pathology: an official journal of the United States and Canadian Academy of Pathology, Inc 24: 209-231. doi: 10.1038/modpathol.2010.205.

10. Hammond M E, Hayes D F, Dowsett M, Allred D C, Hagerty K L, Badve S, Fitzgibbons P L, Francis G, Goldstein N S, Hayes M, Hicks D G, Lester S, Love R, Mangu P B, McShane L, Miller K, Osborne C K, Paik S, Perlmutter J, Rhodes A, Sasano H, Schwartz J N, Sweep F C, Taube S, Torlakovic E E, Valenstein P, Viale G, Visscher D, Wheeler T, Williams R B, Wittliff J L, Wolff A C, American Society of Clinical O, College of American P (2010) American Society of Clinical Oncology/College of American Pathologists guideline recommendations for immunohistochemical testing of estrogen and progesterone receptors in breast cancer (unabridged version). Archives of pathology & laboratory medicine 134: e48-72. doi: 10.1043/1543-2165-134.7.e48.

11. Harbeck N, Nimmrich I, Hartmann A, Ross J S, Cufer T, Grutzmann R, Kristiansen G, Paradiso A, Hartmann O, Margossian A, Martens J, Schwope I, Lukas A, Muller V, Milde-Langosch K, Nahrig J, Foekens J, Maier S, Schmitt M, Lesche R (2008) Multicenter study using paraffin-embedded tumor tissue testing PITX2 DNA methylation as a marker for outcome prediction in tamoxifen-treated, node-negative breast cancer patients. Journal of clinical oncology: official journal of the American Society of Clinical Oncology 26: 5036-5042. doi: 10.1200/JCO.2007.14.1697.

12. Hartmann O, Spyratos F, Harbeck N, Dietrich D, Fassbender A, Schmitt M, Eppenberger-Castori S, Vuaroqueaux V, Lerebours F, Welzel K, Maier S, Plum A, Niemann S, Foekens J A, Lesche R, Martens J W (2009) DNA methylation markers predict outcome in node-positive, estrogen receptor-positive breast cancer with adjuvant anthracycline-based chemotherapy. Clinical cancer research: an official journal of the American Association for Cancer Research 15: 315-323. doi: 10.1158/1078-0432.CCR-08-0166.

13. Hirose H, Ishii H, Mimori K, Tanaka F, Takemasa I, Mizushima T, Ikeda M, Yamamoto H, Sekimoto M, Doki Y, Mori M (2011) The significance of PITX2 overexpression in human colorectal cancer. Annals of surgical oncology 18: 3005-3012. doi: 10.1245/s10434-011-1653-z.

14. Hothorn T (2011) maxstat: Maximally Selected Rank Statistics. In: R package version 0.7-14.

15. Jovanovic J, Ronneberg J A, Tost J, Kristensen V (2010) The epigenetics of breast cancer. Molecular oncology 4: 242-254. doi: 10.1016/j.molonc.2010.04.002.

16. Khasraw M, Bell R, Dang C (2012) Epirubicin: is it like doxorubicin in breast cancer? A clinical review. Breast 21: 142-149. doi: 10.1016/j.breast.2011.12.012.

17. Kioussi C, Briata P, Baek S H, Rose D W, Hamblet N S, Herman T, Ohgi K A, Lin C, Gleiberman A, Wang J, Brault V, Ruiz-Lozano P, Nguyen H D, Kemler R, Glass C K, Wynshaw-Boris A, Rosenfeld M G (2002) Identification of a Wnt/Dvl/beta-Catenin-->Pitx2 pathway mediating cell-type-specific proliferation during development. Cell 111: 673-685.

18. Kreike B, van Kouwenhove M, Horlings H, Weigelt B, Peterse H, Bartelink H, van de Vijver M J (2007) Gene expression profiling and histopathological characterization of triple-negative/basal-like breast carcinomas. Breast cancer research: BCR 9: R65. doi: 10.1186/bcr1771.

19. Lee W K, Chakraborty P K, Thevenod F (2013) Pituitary homeobox 2 (PITX2) protects renal cancer cell lines against doxorubicin toxicity by transcriptional activation of the multidrug transporter ABCB1. International journal of cancer. Journal international du cancer 133: 556-567. doi: 10.1002/ijc.28060.

20. Luu H H, Zhang R, Haydon R C, Rayburn E, Kang Q, Si W, Park J K, Wang H, Peng Y, Jiang W, He T C (2004) Wnt/beta-catenin signaling pathway as a novel cancer drug target. Current cancer drug targets 4: 653-671.

21. Maier S, Nimmrich I, Koenig T, Eppenberger-Castori S, Bohlmann I, Paradiso A, Spyratos F, Thomssen C, Mueller V, Nahrig J, Schittulli F, Kates R, Lesche R, Schwope I, Kluth A, Marx A, Martens J W, Foekens J A, Schmitt M, Harbeck N, European Organisation for R, Treatment of Cancer PathoBiology g (2007) DNA-methylation of the homeodomain transcription factor PITX2 reliably predicts risk of distant disease recurrence in tamoxifen-treated, node-negative breast cancer patients—Technical and clinical validation in a multi-centre setting in collaboration with the European Organisation for Research and Treatment of Cancer (EORTC) PathoBiology group. European journal of cancer 43: 1679-1686. doi: 10.1016/j.ejca.2007.04.025.

22. McShane L M, Altman D G, Sauerbrei W, Taube S E, Gion M, Clark G M, Statistics Subcommittee of the NCIEWGoCD (2005) REporting recommendations for tumor MARKer prognostic studies (REMARK). Nature clinical practice. Oncology 2: 416-422.

23. Mersin H, Yildirim E, Berberoglu U, Gulben K (2008) The prognostic importance of triple negative breast carcinoma. Breast 17: 341-346. doi: 10.1016/j.breast.2007.11.031.

24. Moreno C S, Evans C O, Zhan X, Okor M, Desiderio D M, Oyesiku N M (2005) Novel molecular signaling and classification of human clinically nonfunctional pituitary adenomas identified by gene expression profiling and proteomic analyses. Cancer research 65: 10214-10222. doi: 10.1158/0008-5472. CAN-05-0884.

25. Nimmrich I, Sieuwerts A M, Meijer-van Gelder M E, Schwope I, Bolt-de Vries J, Harbeck N, Koenig T, Hartmann O, Kluth A, Dietrich D, Magdolen V, Portengen H, Look M P, Klijn J G, Lesche R, Schmitt M, Maier S, Foekens J A, Martens J W (2008) DNA hypermethylation of PITX2 is a marker of poor prognosis in untreated lymph node-negative hormone receptor-positive breast cancer patients. Breast cancer research and treatment 111: 429-437. doi: 10.1007/s10549-007-9800-8.

26. Novak P Jensen T, Oshiro M M, Watts G S, Kim C J, Futscher B W (2008) Agglomerative epigenetic aberrations are a common event in human breast cancer. Cancer research 68: 8616-8625. doi: 10.1158/0008-5472.CAN-08-1419.

27. Oakman C, Moretti E, Galardi F, Biagioni C, Santarpia L, Biganzoli L, Di Leo A (2011) Adjuvant systemic treatment for individual patients with triple-negative breast cancer. Breast 20 Suppl 3: S135-141. doi: 10.1016/S0960-9776(11)70311-3.

28. Perou C M, Sorlie T, Eisen M B, van de Rijn M, Jeffrey S S, Rees C A, Pollack J R, Ross D T, Johnsen H, Akslen L A, Fluge O, Pergamenschikov A, Williams C, Zhu S X, Lonning P E, Borresen-Dale A L, Brown P O, Botstein D (2000) Molecular portraits of human breast tumours. Nature 406: 747-752. doi: 10.1038/35021093.

29. Prat A, Parker J S, Karginova O, Fan C, Livasy C, Herschkowitz J I, He X, Perou C M (2010) Phenotypic and molecular characterization of the claudin-low intrinsic subtype of breast cancer. Breast cancer research: BCR 12: R68. doi: 10.1186/bcr2635.

30. Schatz P, Dietrich D, Koenig T, Burger M, Lukas A, Fuhrmann I, Kristiansen G, Stoehr R, Schuster M, Lesche R, Weiss G, Corman J, Hartmann A (2010) Development of a diagnostic microarray assay to assess the risk of recurrence of prostate cancer based on PITX2 DNA methylation. The Journal of molecular diagnostics: JMD 12: 345-353. doi: 10.2353/jmoldx.2010.090088.

31. Schmitt M, Mengele K, Schueren E, Sweep F C, Foekens J A, Brunner N, Laabs J, Malik A, Harbeck N, European Organisation for R, Treatment of Cancer Pathobiology G (2007) European Organisation for Research and Treatment of Cancer (EORTC) Pathobiology Group standard operating procedure for the preparation of human tumour tissue extracts suited for the quantitative analysis of tissue-associated biomarkers. European journal of cancer 43: 835-844. doi: 10.1016/j.ejca.2007.01.008.

32. Shen C, Huang Y, Liu Y, Wang G, Zhao Y, Wang Z, Teng M, Wang Y, Flockhart D A, Skaar T C, Yan P, Nephew K P, Huang T H, Li L (2011) A modulated empirical Bayes model for identifying topological and temporal estrogen receptor alpha regulatory networks in breast cancer. BMC systems biology 5: 67. doi: 10.1186/1752-0509-5-67.

33. Sorlie T, Perou C M, Tibshirani R, Aas T, Geisler S, Johnsen H, Hastie T, Eisen M B, van de Rijn M, Jeffrey S S, Thorsen T, Quist H, Matese J C, Brown P O, Botstein D, Lonning PE, Borresen-Dale AL (2001) Gene expression patterns of breast carcinomas distinguish tumor subclasses with clinical implications. Proceedings of the National Academy of Sciences of the United States of America 98: 10869-10874. doi: 10.1073/pnas.191367098.

34. Tan D S, Marchio C, Jones R L, Savage K, Smith I F, Dowsett M, Reis-Filho J S (2008) Triple-negative breast cancer: molecular profiling and prognostic impact in adjuvant anthracycline-treated patients. Breast cancer research and treatment 111: 27-44. doi: 10.1007/s10549-007-9756-8.

35. Team RDC (2012) R: A language and environment for statistical computing. In:R Foundation for Statistical Computing, Vienna, Austria.

36. Theriault R L, Litton J K, Mittendorf E A, Chen H, Meric-Bernstam F, Chavez-Macgregor M, Morrow P K, Woodward W A, Sahin A, Hortobagyi G N, Gonzalez-Angulo A M (2011) Age and survival estimates in patients who have node-negative T1ab breast cancer by breast cancer subtype. Clinical breast cancer 11: 325-331. doi: 10.1016/j.clbc.2011.05.002.

37. Thike A A, Cheok P Y, Jara-Lazaro A R, Tan B, Tan P, Tan P H (2010) Triple-negative breast cancer: clinicopathological characteristics and relationship with basal-like breast cancer. Modern pathology : an official journal of the United States and Canadian Academy of Pathology, Inc 23 :123-133. doi: 10.1038/modpathol.2009.145.

38. Vaklavas C, Forero-Torres A (2011) How do I treat “triple-negative” disease. Current treatment options in oncology 12 :369-388. doi: 10.1007/s11864-011-0168-y.

39. Vela I, Morrissey C, Zhang X, Chen S, Corey E, Strutton G M, Nelson C C, Nicol D L, Clements J A, Gardiner E M (2013) PITX2 and non-canonical Wnt pathway interaction in metastatic prostate cancer. Clinical & experimental metastasis. doi: 10.1007/s10585-013-9620-7.

40. Wolff A C, Hammond M E, Schwartz J N, Hagerty K L, Allred D C, Cote R J, Dowsett M, Fitzgibbons PL, Hanna W M, Langer A, McShane L M, Paik S, Pegram M D, Perez E A, Press M F, Rhodes A, Sturgeon C, Taube SE, Tubbs R, Vance G H, van de Vijver M, Wheeler T M, Hayes D F, American Society of Clinical Oncology/College of American P (2007) American Society of Clinical Oncology/College of American Pathologists guideline recommendations for human epidermal growth factor receptor 2 testing in breast cancer. Archives of pathology & laboratory medicine 131: 18-43. doi: 10.1043/1543-2165(2007)131[18:ASOCCO]2.0.CO;2.

1. A method for predicting a triple-negative breast cancer (TNBC) patient's response to anthracycline-containing polychemotherapy, said method comprising:
i) contacting genomic DNA isolated from a biological sample of said TNBC patient with at least one reagent, or series of reagents, that distinguishes between methylated and non-methylated CpG dinucleotides;
ii) determining, based on such contacting of i), a methylation state of at least one CpG dinucleotide sequence of a paired-like homeodomain transcription factor 2 (PITX2) gene and/or of one or several regulatory sequences thereof within said biological sample, and iii) making a prediction of said TNBC patient's response to anthracycline-containing polychemotherapy based on the determined methylation state.

2. The method according to claim 1, wherein said polychemotherapy is adjuvant polychemotherapy.

3. The method according to claim 1, wherein said prediction of said TNBC patient's response to anthracycline-containing polychemotherapy is based on whether the determined methylation state of said PITX2 gene and/or of one or several regulatory sequences thereof exceeds a defined threshold, wherein said prediction is made in terms of one or several parameters selected from disease-free survival (DFS), metastasis-free survival (MFS) and overall survival (OS), respectively, and wherein said prediction is negative, if the determined methylation state is equal to or does not exceed said threshold, and wherein said prediction is positive, if said determined methylation state does exceed said threshold.

4. The method according to claim 1, wherein said contacting in step i) comprises contacting genomic DNA isolated from said biological sample obtained from said patient with at least one reagent, or series of reagents that distinguishes between methylated and non-methylated CpG dinucleotides within at least one target region of the genomic DNA, wherein the target region comprises or hybridizes under stringent conditions to a sequence of at least 16 contiguous nucleotides of the PITX2 gene and/or of regulatory sequences thereof, wherein said contiguous nucleotides comprise at least one CpG dinucleotide sequence.

5. The method according to claim 1, wherein said one or more reagents comprise bisulfite, hydrogen sulfite, disulfite or combinations thereof.

6. The method according to claim 1, wherein the step of determining the methylation state is performed by oligonucleotide hybridization analysis, methylation-sensitive single-nucleotide primer extension (Ms-SNuPE), sequencing, quantitative real time PCR or oligonucleotide array analysis.

7. The method according to claim 1, wherein said determination of said methylation state in step ii) is or comprises the determination of a methylation score of said PITX2 gene or of one or several regulatory sequences thereof in said biological sample, and wherein prediction of said TNBC patient's response to said anthracycline-containing polychemotherapy is based on whether the methylation score in said biological sample exceeds a defined threshold methylation score, wherein said prediction is made in terms of one or several parameters selected from disease-free survival (DFS), metastasis-free survival (MFS) and overall survival (OS), and wherein said prediction is negative, if the methylation score of said sample is equal to or does not exceed said defined threshold methylation score, and wherein said prediction is positive, if said methylation score of said sample does exceed said defined threshold methylation score,
wherein said methylation score in said biological sample is determined according to the formula:
$$methylation score in biological sample = \frac{100}{1 + {2}^{( CT methylated - CT unmethylated )}}$$
wherein “CT methylated” is the cycle threshold value of methylated PITX2 and/or of methylated regulatory sequence(s) thereof,
and wherein “CT unmethylated” is the cycle threshold value of unmethylated PITX2 and/or of unmethylated regulatory sequence(s) thereof.

8. The method according to claim 1, comprising the steps:
i′) isolating genomic DNA from a biological sample taken from said patient and treating said genomic DNA, or a fragment thereof, with one or more reagents, in order to convert 5-position unmethylated cytosine bases to uracil or to another base that is detectably dissimilar to cytosine in terms of hybridization properties, thus producing treated genomic DNA; contacting said treated genomic DNA or said treated fragment thereof, with an amplification enzyme and at least two primers comprising, in each case, a contiguous sequence of at least 16 nucleotides in length that is complementary to, or hybridizes under stringent conditions to a sequence of the PITX2 gene and/or to one or several of its regulatory sequences thereof, and/or to complements thereof, wherein said at least two primers hybridize to said sequence(s) only if such sequence(s) has(have) been treated with said one or more reagents, such that the treated DNA or a fragment thereof is amplified to produce one or more amplificates, and wherein during amplification there are additionally at least two PITX2-specific probes present that distinguish between methylated and unmethylated PITX2 sequences, said at least two PITX2-specific probes producing two signals being indicative of methylated and unmethylated PITX2 sequences, respectively, said signals being proportional to the amount of methylated und unmethylated PITX2-sequences present in said biological sample, respectively,
ii′) determining, based on said signals of methylated und unmethylated PITX2-sequences a methylation score of said PITX2 gene in said biological sample,
wherein said methylation score in said biological sample is determined according to the formula
$$methylation score in biological sample = \frac{100}{1 + {2}^{( CT methylated - CT unmethylated )}}$$
wherein “CT methylated” is the cycle threshold value of methylated PITX2 and/or of methylated regulatory sequence(s) thereof,
and wherein “CT unmethylated” is the cycle threshold value of unmethylated PITX2 and/or of unmethyated regulatory sequence(s) thereof, and
iii′) making a prediction of said TNBC patient's response to anthracycline-containing polychemotherapy based on said determined methylation score in said biological sample, and wherein prediction of said TNBC patient's response to said anthracycline-containing polychemotherapy is based on whether the methylation score in said biological sample exceeds a defined threshold methylation score, wherein said prediction is made in terms of one or several parameters selected from disease-free survival (DFS), metastasis-free survival (MFS) and overall survival (OS), and wherein said prediction is negative, if the methylation score of said sample is equal to or does not exceed said defined threshold methylation score, and wherein said prediction is positive, if said methylation score of said sample does exceed said defined threshold methylation score.

9. The method according claim 1, further comprising
iv) determining a suitable treatment regimen for said patient, wherein such suitable treatment regimen for said patient is a treatment with polychemotherapy including one or several anthracyclines if said prediction of step iii) or iii′) is positive, and said suitable treatment regimen is a therapy excluding anthracycline treatment, if said prediction is negative.

10. The method according to claim 7, wherein said defined threshold methylation score is in the range of from 4-10.

11. The method according to claim 1, wherein said biological sample is selected from the group consisting of presurgical core biopsies, biopsies taken at time of primary surgery, circulating peripheral breast cancer tumor cells, tumor-afflicted lymph nodes, metastases, nipple aspirate, blood, serum, plasma, urine or any other bodily fluid, and combinations thereof.

12. The method according to claim 1, wherein the PITX2 gene has a sequence represented by SEQ ID NO:1 or SEQ ID NO:2.

13. The method according to claim 8, wherein said sequence which said at least two primers are complementary to or hybridize thereto has a sequence represented by SEQ ID NO: 3 and/or 4, and/or wherein said sequence of the PITX2 gene before treatment with said one or more reagent(s) has a sequence represented by SEQ ID NO:5, and/or wherein said at least two primers have sequences represented by SEQ ID NO: 6 and 7; and/or wherein said at least two PITX2-specific probes have sequences represented by SEQ ID NO:8 and SEQ ID NO:9.

14.. The method according to claim 8, wherein said at least two PITX2-specific probes are Taqman hydrolysis probes that distinguish between methylated and unmethylated PITX2 sequences, wherein a first Taqman hydrolysis probe is specific for methylated PITX2 sequence(s) and produces a first signal upon amplification of methylated PITX2 sequence(s), and wherein a second Taqman hydrolysis probe is specific for unmethylated PITX2 sequence(s) and produces a second signal different from said first signal, upon amplification of unmethylated PITX2 sequence(s), said first and said second signal preferably being fluorescence signals.

15. (canceled)

16. The use method according to claim 8, wherein said PITX2 gene, said PITX2 regulatory sequence, said stretch of at least 16 contiguous nucleotides of the foregoing sequences, said complementary sequence thereto, or said sequence that hybridizes under stringent conditions to any of the foregoing sequences, is selected from any of the sequences represented by SEQ ID NO:1-9.
